(19) United States
(12) Patent Application Publication
(10) Pub. No.: US 2025/0186202 A1
(43) Pub. Date: Jun. 12, 2025

(54) PROSTHETIC HEART VALVE

(71) Applicant: EDWARDS LIFESCIENCES CORPORATION, Irvine, CA (US)

(72) Inventors: Nikolai Gurovich, Hadera (IL); Tamir S. Levi, Zikhron Yaakov (IL); Michael Bukin, Pardes Hanna (IL); Karin Lavon, Tel-Aviv (IL)

(73) Assignee: EDWARDS LIFESCIENCES CORPORATION, Irvine, CA (US)

(21) Appl. No.: 19/061,017

(22) Filed: Feb. 24, 2025

Related U.S. Application Data
(63) Continuation of application No. PCT/US2023/031121, filed on Aug. 25, 2023.
(60) Provisional application No. 63/401,538, filed on Aug. 26, 2022.

(51) Int. Cl. A61F 2/24

(52) U.S. Cl. CPC A61F 2/2418; A61F 2220/0025; A61F 2250/0037

(57) ABSTRACT

Expandable frames for prosthetic heart valves are disclosed. As one example, a prosthetic valve can include a frame comprising interconnected angled struts defining rows of cells between first and second ends of the frame, the struts including a first row of struts at the first end, a second row of struts, and a third row of struts. A first row of first cells is disposed at the first end and defined by the first and second rows of struts and a second row of second cells is disposed adjacent to the first row of first cells and defined by the second and third rows of struts, each first cell wider than each second cell, and the second row of struts comprising a plurality of free apex regions, each connecting adjacent ends of a respective pair of angled struts and having a first surface with a constant convex curvature.

### CROSS-REFERENCE TO RELATED APPLICATIONS

This application is a continuation of PCT Application No. PCT/US2023/031121, filed Aug. 25, 2023, which claims the benefit of U.S. Provisional Patent Application No. 63/401,538, filed Aug. 26, 2022, the entire contents of each of which are incorporated by reference herein.

### FIELD

The present disclosure relates to expandable prosthetic heart valves, including frames for prosthetic heart valves.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (e.g., stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (e.g., through a femoral artery and the aorta) until the prosthetic valve reaches the implantation site in the heart. The prosthetic valve is then expanded to its functional size, for example, by inflating a balloon on which the prosthetic valve is mounted, actuating a mechanical actuator that applies an expansion force to the prosthetic valve, or by deploying the prosthetic valve from a sheath of the delivery apparatus so that the prosthetic valve can self-expand to its functional size.

Most expandable prosthetic heart valves comprise a radially expandable and compressible cylindrical metal frame or stent and prosthetic leaflets mounted inside the frame. In some examples, prosthetic heart valves can include an inner skirt disposed around an inside of the frame, with the leaflets secured to the inner skirt.

In some examples, a prosthetic heart valve can be implanted within the aortic root, which includes the right and left coronary ostia and is defined between the native aortic valve annulus and the sinotubular junction (STJ). The prosthetic heart valve can be implanted either within the native aortic valve or within a previously implanted prosthetic heart valve (e.g., previously implanted via a valve-in-valve (ViV) procedure). However, during such implantations, there can be a risk of at least partially blocking the coronary ostia either by native aortic valve leaflets that are pushed sideways during expansion of the prosthetic heart valve, by prosthetic leaflets of a previously implanted prosthetic heart valve that are similarly pushed sideways during expansion of a new prosthetic heart valve during a ViV procedure, and/or by the overlapping frames of the two valves following the ViV procedure. As a result, access to the coronary arteries for later interventions (such as with a catheter) may be made more difficult.

Accordingly, a need exists for improved frame designs for prosthetic heart valves.

### SUMMARY

Described herein are prosthetic heart valves, delivery apparatuses, and methods for implanting prosthetic heart valves. In particular, described herein are examples of radially expandable and compressible frames for prosthetic heart valves. The frame of a prosthetic heart valve can comprise a plurality of interconnected struts that define a plurality of rows of cells arranged between an outflow end and an inflow end of the frame. In some examples, a first row of cells disposed at the outflow end can have fewer cells that subsequent rows of the frame such that the cells in the first row of cells have a width (in a circumferential direction) that is larger than cells in subsequent rows. As a result, cells in a second row of cells that are adjacent to the first row of cells can have apices that are exposed (or free) and unattached to additional struts defining the first row of cells. In some examples, these apices can have a curved profile with a constant convex curvature between the angled struts or strut portions to which they are connected, similar to the apices or apex regions of the first row of cells at the outflow end of the frame. As a result, these apices or apex regions can be more atraumatic and not interfere with the leaflets of the prosthetic heart valve as the leaflets open and close during operation of the prosthetic heart valve. In some examples, an inner skirt can be disposed around an inner surface of the frame, with an outflow edge of the inner skirt secured to struts forming the first and second row of cells and disposed below (upstream of, or toward the inflow end of the frame) the exposed apices or apex regions. As such, the devices and methods disclosed herein can, among other things, overcome one or more of the deficiencies of typical prosthetic heart valves.

A prosthetic heart valve can comprise a frame and a valvular structure coupled to the frame. In addition to these components, a prosthetic heart valve can further comprise one or more of the components disclosed herein.

In some examples, the frame can comprise a plurality of interconnected angled struts defining a plurality of circumferentially extending rows of cells arranged between an outflow end and an inflow end of the frame, where the plurality of interconnected angled struts is arranged to form a plurality of circumferentially extending rows of struts, including a first row of struts at the outflow end of the frame, a second row of struts upstream of the first row of struts, and a third row of struts upstream of the second row of struts.

In some examples, the plurality of circumferentially extending rows of cells comprises a first row of first cells disposed at the outflow end and at least partially defined by the first and second rows of struts, and a second row of second cells disposed upstream of the first row of first cells and at least partially defined by the second and third rows of struts, where each first cell of the first row of first cells has a first width that is larger than a second width of each second cell of the second row of second cells.

In some examples, the second row of struts comprises a plurality of free apex regions, and each free apex region connects adjacent ends of a respective pair of angled struts of the second row of struts together.

In some examples, each free apex region has a first surface facing in a downstream direction and an opposing second surface facing in an upstream direction, where the first surface has a constant convex curvature extending between the adjacent ends of the respective pair of angled struts.

In some examples, the plurality of interconnected struts further comprises a plurality of axially extending struts spaced circumferentially apart around the frame and connected to the first row of struts. A first portion of angled struts of the second row of struts are each directly connected to a respective axially extending strut of the plurality of axially extending struts. A second portion of angled struts of the second row of struts form pairs of angled struts which are connected together by a free apex region that is unattached to the plurality of axially extending struts.

In some examples, the prosthetic heart valve can comprise an inner skirt disposed around an inner surface of the frame, wherein an outflow edge portion of the inner skirt is secured to the second row of struts, and wherein at each free apex region, the outflow edge portion is disposed upstream of the free apex region.

In some examples, a prosthetic heart valve comprises a radially expandable and compressible annular frame comprising a plurality of interconnected angled struts defining a plurality of circumferentially extending rows of cells arranged between an outflow end and an inflow end of the frame, wherein the plurality of interconnected angled struts is arranged to form a plurality of circumferentially extending rows of struts, including a first row of struts at the outflow end of the frame, a second row of struts upstream of the first row of struts, and a third row of struts upstream of the second row of struts. The plurality of circumferentially extending rows of cells comprises a first row of first cells disposed at the outflow end and at least partially defined by the first and second rows of struts, and a second row of second cells disposed upstream of the first row of first cells and at least partially defined by the second and third rows of struts, where each first cell of the first row of first cells has a first width that is larger than a second width of each second cell of the second row of second cells. The second row of struts comprises a plurality of free apex regions, and each free apex region connects adjacent ends of a respective pair of angled struts of the second row of struts together and has a first surface facing in a downstream direction and an opposing second surface facing in an upstream direction, where the first surface has a constant convex curvature extending between the adjacent ends of the respective pair of angled struts.

In some examples, a prosthetic heart valve comprises a radially expandable and collapsible annular frame comprising a plurality of interconnected struts defining a plurality of circumferentially extending rows of cells arranged between an outflow end and an inflow end of the frame. The plurality of interconnected struts comprises a plurality of circumferentially extending rows of angled struts including a first row of struts at the outflow end of the frame, a second row of struts upstream of the first row of struts, and a third row of struts upstream of the second row of struts, and a plurality of axial struts spaced circumferentially apart around the frame and extending between the first row of struts and the second row of struts. The plurality of circumferentially extending rows of cells comprises a first row of first cells disposed at the outflow end and at least partially defined by the first and second rows of struts and the plurality of axial struts, and a second row of second cells disposed upstream of the first row of first cells and at least partially defined by the second and third rows of struts. Each first cell of the first row of first cells has a first width that is larger than a second width of each second cell of the second row of second cells. The second row of second cells comprises a first portion of second cells that are defined by first pairs of angled struts of the second row of struts that are directly connected to the plurality of axial struts at their adjacent ends, and a second portion of second cells that are defined by second pairs of angled struts of the second row of struts and a plurality of free apex regions. Each free apex region connects adjacent ends of a respective second pair of angled struts together and has a first surface facing in a downstream direction and an opposing second surface facing in an upstream direction, where the first surface has a constant convex curvature extending between the adjacent ends of the respective second pair of angled struts, and wherein the plurality of free apex regions is unattached to the plurality of axial struts.

In some examples, a prosthetic heart valve comprises a radially expandable and collapsible annular frame comprising a plurality of interconnected struts defining a plurality of circumferentially extending rows of cells arranged between an outflow end and an inflow end of the frame. The plurality of interconnected struts comprises a circumferentially extending row of first struts defining the outflow end, each first strut comprising two angled strut portions interconnected by an outflow apex region, where the outflow apex region curves between the two angled strut portions and has a narrowed width relative to a width of the two angled struts portions. The plurality of interconnected struts further comprises a plurality of axially extending struts spaced circumferentially apart around the frame and connected to the row of first struts, and a circumferentially extending row of angled second struts disposed upstream of the row of first struts. A first portion of second struts of the row of angled second struts are each directly connected to a respective axially extending strut of the plurality of axially extending struts. A second portion of second struts of the row of angled second struts form pairs of second struts which are connected together by a free apex region that is unattached to the plurality of axially extending struts, where the free apex region of each respective pair of second struts has a first surface facing in a downstream direction and an opposing second surface facing in an upstream direction, where the first surface curves between the respective pair of second struts, and where the second surface depresses inward toward the first surface such that a width of the free apex region between the first and second surfaces is less than a width of the respective pair of second struts. The plurality of interconnected struts further comprises a circumferentially extending row of angled third struts, where the row of first struts, the axially extending struts, and the row of angled second struts form a first row of cells of the plurality of rows of cells disposed at the outflow end, where the row of angled second struts and the row of angled third struts form a second row of cells of the plurality of rows of cells that are disposed adjacent to the first row of cells, and where a first width of each cell of the first row of cells is wider than a second width of each cell of the second row of cells. The prosthetic heart valve further comprises a plurality of leaflets secured on an inside of the frame and configured to open and close in order to regulate a flow of blood through the prosthetic heart valve, from the inflow end to the outflow end of the frame, where each free apex region of the row of angled second struts is disposed at a level of a portion of the plurality of leaflets that open and close during operation of the prosthetic heart valve.

In some examples, a prosthetic heart valve comprises a radially expandable and compressible annular frame comprising a plurality of interconnected angled struts defining a plurality of circumferentially extending rows of cells arranged between a first end and a second end of the frame, where the plurality of interconnected angled struts is arranged to form a plurality of circumferentially extending rows of struts, including a first row of struts at the first end of the frame, a second row of struts disposed adjacent to the first row of struts, and a third row of struts disposed adjacent to the second row of struts, the second row of struts disposed between the first and third row of struts. The plurality of circumferentially extending rows of cells comprises a first row of first cells disposed at the first end and at least partially defined by the first and second rows of struts, and a second row of second cells disposed adjacent to the first row of first cells and at least partially defined by the second and third rows of struts, where each first cell of the first row of first cells has a first width that is larger than a second width of each second cell of the second row of second cells. The second row of struts comprises a plurality of free apex regions, and each free apex region connects adjacent ends of a respective pair of angled struts of the second row of struts together and has a first surface facing the first end of the frame and an opposing second surface facing the second end of the frame, where the first surface has a constant convex curvature extending between the adjacent ends of the respective pair of angled struts.

In some examples, a prosthetic heart valve comprises a radially expandable and compressible annular frame comprising a plurality of interconnected struts defining a plurality of circumferentially extending rows of cells arranged between an inflow end and an outflow end of the frame. The plurality of interconnected struts comprises a circumferentially extending row of outflow struts defining the outflow end, where each outflow strut comprises two angled strut portions interconnected by an apex region, where each apex region curves between a corresponding pair of two angled strut portions and has a narrowed width and a length that extends along at least 25% of a total length of the outflow strut, where the narrowed width is smaller than a width of the two angled strut portions. The plurality of interconnected struts further comprises a circumferentially extending row of angled first struts disposed upstream of the row of outflow struts, where the row of outflow struts and the row of angled first struts at least partially form a first row of cells of the plurality of circumferentially extending rows of cells disposed at the outflow end, and where each cell of the first row of cells has a first width that is larger than a second width of cells of remaining rows of cells of the plurality of circumferentially extending rows of cells.

In some examples, a prosthetic heart valve comprises a radially expandable and compressible annular frame comprising a plurality of interconnected angled struts defining a plurality of circumferentially extending rows of cells arranged between a first end and a second end of the frame, where the plurality of interconnected angled struts is arranged to form a plurality of circumferentially extending rows of struts, including a first row of struts at the first end of the frame, a second row of struts disposed adjacent to the first row of struts, and a third row of struts disposed adjacent to the second row of struts, the second row of struts disposed between the first and third row of struts. The plurality of circumferentially extending rows of cells comprises: a first row of first cells disposed at the first end and at least partially defined by the first and second rows of struts; and a second row of second cells disposed adjacent to the first row of first cells and at least partially defined by the second and third rows of struts, where each first cell of the first row of first cells has a first width that is larger than a second width of each second cell of the second row of second cells. The second row of struts comprises a plurality of free apices, and each free apex connects adjacent ends of a respective pair of angled struts of the second row of struts together. The prosthetic heart valve further comprises an inner skirt disposed around an inner surface of the frame, where a first edge portion of the inner skirt is secured to the second row of struts, where at each free apex the first edge portion is disposed away from the free apex toward the second end of the frame, and where a second edge portion of the inner skirt is disposed at the second end of the frame.

In some examples, a prosthetic heart valve comprises a radially expandable and compressible annular frame comprising a plurality of interconnected angled struts arranged to form a plurality of circumferentially extending rows of struts, including a first row of struts, a second row of struts downstream of the first row of struts, and a third row of struts downstream of the second row of struts. The prosthetic heart valve further comprises an inner skirt disposed around an inner surface of the frame. The inner skirt comprises an inflow edge and an outflow edge, where the outflow edge is stitched to the struts of the second row of struts and comprises a plurality of peaks spaced apart from each other in a circumferential direction, where the peaks are aligned with respective apices of the second row of struts, and where at least one peak has a straight edge that is spaced from the respective apex toward an inflow end of the frame.

In some examples, a prosthetic heart valve comprises a radially expandable and compressible annular frame comprising a plurality of interconnected angled struts defining a plurality of circumferentially extending rows of cells arranged between an outflow end and an inflow end of the frame, where the plurality of interconnected angled struts is arranged to form a plurality of circumferentially extending rows of struts, including a first row of struts at the outflow end of the frame, a second row of struts upstream of the first row of struts, and a third row of struts upstream of the second row of struts. The plurality of circumferentially extending rows of cells comprises: a first row of first cells disposed at the outflow end and at least partially defined by the first and second rows of struts and axially extending struts interconnecting struts of the first and second rows of struts; and a second row of second cells disposed upstream of the first row of first cells and at least partially defined by the second and third rows of struts. The second row of struts comprises a plurality of free apex regions that are not connected to struts of the first row of struts by axially extending struts, and where each free apex region connects adjacent ends of a respective pair of angled struts of the second row of struts together and has a first surface facing in a downstream direction, an opposing second surface facing in an upstream direction, and a width measured from the first surface to the second surface, where the width is less than width of the struts connected by the free apex region.

In some examples, a prosthetic heart valve comprises one or more of the components recited in Examples 1-38, 55-124, and 126 below.

An assembly can comprise a delivery apparatus, and an implantable prosthetic heart valve that is radially collapsible to a collapsed configuration and radially expandable to an expanded configuration.

In some examples, the delivery apparatus comprises a balloon and the collapsed prosthetic heart valve can be mounted around the balloon and radially expanded to the expanded configuration with the balloon inside a patient's body.

In some examples, the prosthetic heart valve comprises a radially expandable and compressible annular frame comprising a plurality of interconnected angled struts defining a plurality of circumferentially extending rows of cells arranged between an outflow end and an inflow end of the frame.

In some examples, the plurality of interconnected angled struts is arranged to form a plurality of circumferentially extending rows of struts, including a first row of struts at the outflow end of the frame, a second row of struts upstream of the first row of struts, and a third row of struts upstream of the second row of struts. The plurality of circumferentially extending rows of cells comprises a first row of first cells disposed at the outflow end and at least partially defined by the first and second rows of struts, and a second row of second cells disposed upstream of the first row of first cells and at least partially defined by the second and third rows of struts, where each first cell of the first row of first cells has a first width that is larger than a second width of each second cell of the second row of second cells.

In some examples, the second row of struts comprises a plurality of free apex regions, and each free apex region connects together a respective pair of angled struts of the second row of struts.

In some examples, each apex region has a first surface facing the outflow end of the frame and an opposing second surface facing the inflow end of the frame.

In some examples, the first surface forms a single, continuous convex curve from one angled strut of the respective pair of angled struts on a first side of the free apex region to another angled strut of the respective pair of angled struts on an opposite, second side of the free apex region.

In some examples, the prosthetic heart valve can comprise an inner skirt disposed around an inner surface of the frame, wherein an outflow edge portion of the inner skirt is secured to the second row of struts, and wherein at each free apex region, the outflow edge portion is disposed upstream of the free apex region.

In some examples, an assembly comprises a delivery apparatus comprising a balloon, and an implantable prosthetic heart valve that is radially collapsible to a collapsed configuration and radially expandable to an expanded configuration. The prosthetic heart valve comprises a radially expandable and compressible annular frame comprising a plurality of interconnected angled struts defining a plurality of circumferentially extending rows of cells arranged between an outflow end and an inflow end of the frame, where the plurality of interconnected angled struts is arranged to form a plurality of circumferentially extending rows of struts, including a first row of struts at the outflow end of the frame, a second row of struts upstream of the first row of struts, and a third row of struts upstream of the second row of struts. The plurality of circumferentially extending rows of cells comprises a first row of first cells disposed at the outflow end and at least partially defined by the first and second rows of struts, and a second row of second cells disposed upstream of the first row of first cells and at least partially defined by the second and third rows of struts, where each first cell of the first row of first cells has a first width that is larger than a second width of each second cell of the second row of second cells. The second row of struts comprises a plurality of free apex regions, and each free apex region connects together a respective pair of angled struts of the second row of struts and has a first surface facing the outflow end of the frame and an opposing second surface facing the inflow end of the frame. The first surface forms a single, continuous convex curve from one angled strut of the respective pair of angled struts on a first side of the free apex region to another angled strut of the respective pair of angled struts on an opposite, second side of the free apex region. The collapsed prosthetic heart valve can be mounted around the balloon and radially expanded to the expanded configuration with the balloon inside a patient's body.

In some examples, an assembly comprises one or more of the components recited in Examples 39-54 below.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 is a side view of a prosthetic heart valve, according to one example.

FIG. 2 is a side view of a frame of the prosthetic heart valve of FIG. 1.

FIG. 3 is a side view of a portion of the frame of FIG. 2, showing the portion of the frame in a straightened (non-annular) state.

FIG. 4 is a side view of an exemplary delivery apparatus configured to deliver and implant a radially expandable prosthetic heart valve at an implantation site.

FIG. 5A is a side view of a portion of an exemplary frame for a prosthetic heart valve in a radially expanded configuration, the frame comprising a first row of cells that are wider than cells of an adjacent, second row of cells, which results in a portion of cells of the second row of cells having curved apex regions that are unattached to struts forming the first row of cells.

FIG. 5B is a side view of the portion of the frame of FIG. 5A in a radially compressed configuration.

FIG. 6 is a side view of the complete frame of FIG. 5A in a straightened (non-annular) state.

FIG. 7 is a side view of a portion of another exemplary frame for a prosthetic heart valve where angled struts of the frame assume an inwardly bent orientation in the radially compressed state of the frame.

FIG. 8 is a side view of a portion of another exemplary frame for a prosthetic heart valve where angled struts of the frame assume a relatively straight vertical orientation in the radially compressed state of the frame.

FIG. 9 is an interior side view of the portion of the frame of FIG. 5A with an inner skirt disposed on inner surfaces of the struts, the inner skirt having an outflow edge that is arranged below the apex regions that are unattached to struts forming the first row of cells.

FIG. 10 is a cross-sectional view of the frame and inner skirt of FIG. 9, with an outer skirt disposed on an outer surface of the frame, and the outflow edge of the inner skirt folded over itself and arranged upstream of the free apex region of the frame.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (e.g., out of the patient's body), while distal motion of the device is motion of the device away from the user and toward the implantation site (e.g., into the patient's body). The terms “longitudinal” and “axial” refer to an axis extending in the proximal and distal directions, unless otherwise expressly defined.

As used herein, “e.g.” means “for example,” and “i.e.” means “that is.”

### Overview of the Disclosed Technology

As introduced above, prosthetic heart valves can comprise a radially expandable and compressible annular frame and a plurality of leaflets attached to the frame. The frame can include a plurality of rows of cells formed by interconnected struts of the frame. The plurality of rows of cells can include a first row of cells arranged at an outflow end of the frame. In some examples, the cells of the first row of cells are elongated in an axial direction relative to cells of remaining rows of cells of the frame.

Additionally or alternatively, in some examples, in order to further increase a size of the cells of the first row of cells for increased coronary access following implantation, the cells of the first row of cell can be wider in a circumferential direction relative to cells of remaining rows of cells of the frame. For example, in some instances there may be one cell in the first row of cells for every two cells in each remaining row of cells (for example, due to the cells in the first row being twice as wide as the cells in the remaining rows of cells). As a result, cells of a second row of cells disposed adjacent to and connected to the first row of cells can include free apices that are unattached to (additional) struts defining the first row of cells.

Since the leaflets of the prosthetic valve move between a closed state and an open state during operation of the prosthetic valve (when implanted in a patient's body), they may contact (in their open state) these free apices of the second row of cells. In some instances, this can decrease a longevity of the leaflets.

Prosthetic valves disclosed herein can be radially compressible and expandable between a radially compressed state and a radially expanded state. Thus, the prosthetic valves can be crimped on or retained by an implant delivery apparatus in the radially compressed state while being advanced through a patient's vasculature on the delivery apparatus. The prosthetic valve can be expanded to the radially expanded state once the prosthetic valve reaches the implantation site. It is understood that the prosthetic valves disclosed herein may be used with a variety of implant delivery apparatuses and can be implanted via various delivery procedures, examples of which will be discussed in more detail later.

In some instances, when the prosthetic valve is in the radially compressed state and the angled struts of the frame assume a more vertical orientation, the leaflets can become caught between adjacent struts of the frame. This can also decrease a longevity of the leaflets.

Described herein are various examples of frames for prosthetic heart valves comprising a first row of cells disposed at a first end of the frame (for example, an outflow end) that are wider in a circumferential direction than cells of remaining rows of cells of the frame, thereby resulting in exposed or free apices formed by a row of angled struts partially defining a second row of cells that is adjacent to the first row of cells. The free apices can have a first surface facing the outflow end of the frame and an opposing second surface facing the inflow end of the frame, where the first surface has a constant convex curvature extending between adjacent ends of a pair of angled struts of the row of angled struts. In some examples, the free apices or apex regions can have a narrowed width relative to the angled struts to which they are connected. As a result, these free apices or apex regions can be more atraumatic and may not interfere with the leaflets of the prosthetic heart valve as the leaflets open and close during operation of the prosthetic heart valve. As a result, a longevity of the leaflets can be increased.

In some examples, an inner skirt can be disposed around an inner surface of the frame. An outflow edge of the inner skirt can have a zig-zag shape and be secured to the row of struts forming the free apices. However, in some examples, the outflow edge of the inner skirt can be trimmed or arranged below or upstream of the free apices. In some examples, the outflow edge can also be folded over itself (toward the frame), such that its rough edge is tucked away from the leaflets. As a result, a longevity of the leaflets can be increased.

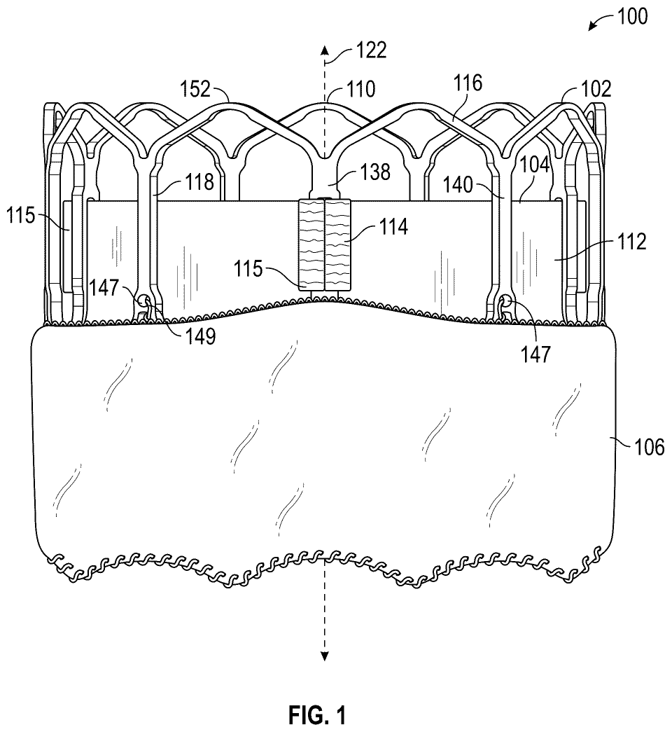

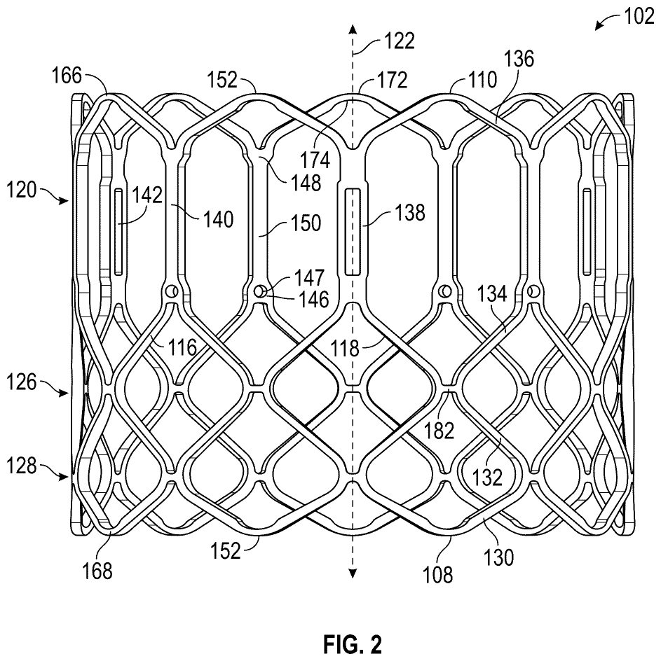

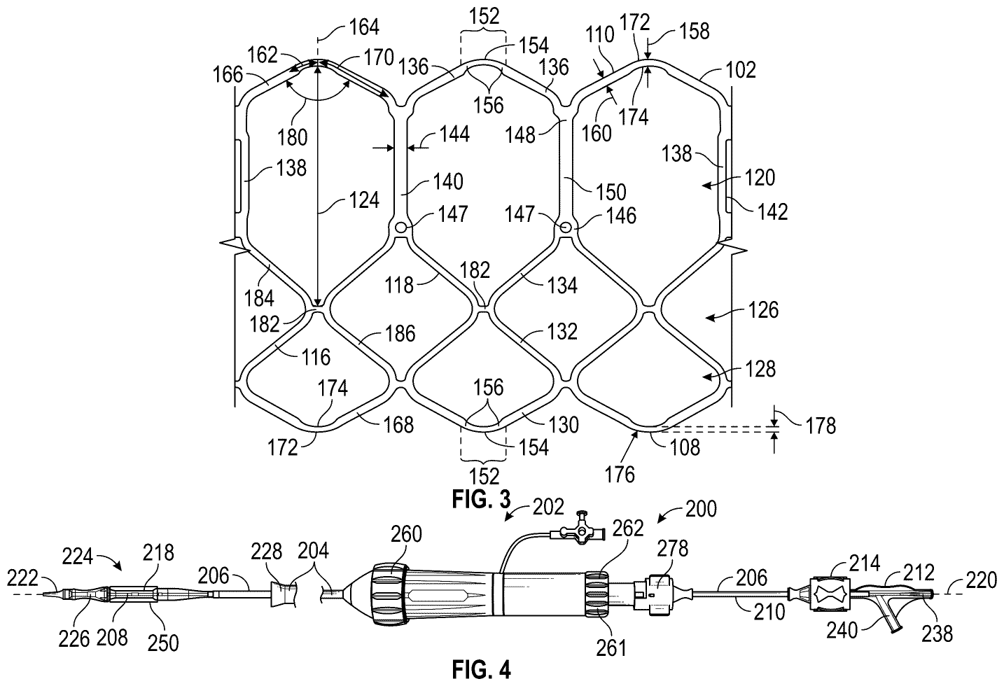

FIG. 1 illustrates an exemplary prosthetic device (e.g., prosthetic heart valve) comprising a frame, leaflets secured on an inside of the frame, and an outer skirt disposed around an outer surface of the frame. In some examples, the frame can comprise a plurality of interconnected and angled struts and apex regions that extend and/or curve between the angled struts at an inflow end and outflow end of the frame, as shown in FIGS. 2 and 3. In some examples, cells of a first row of cells of the frame that are disposed at a first end (for example, an outflow end) of the frame can be elongated in an axial direction relative to cells of remaining rows of cells of the frame (FIGS. 2 and 3). The prosthetic device can be advanced through a patient's vasculature, such as to a native heart valve, by a delivery apparatus, such as the exemplary delivery apparatus shown in FIG. 4.

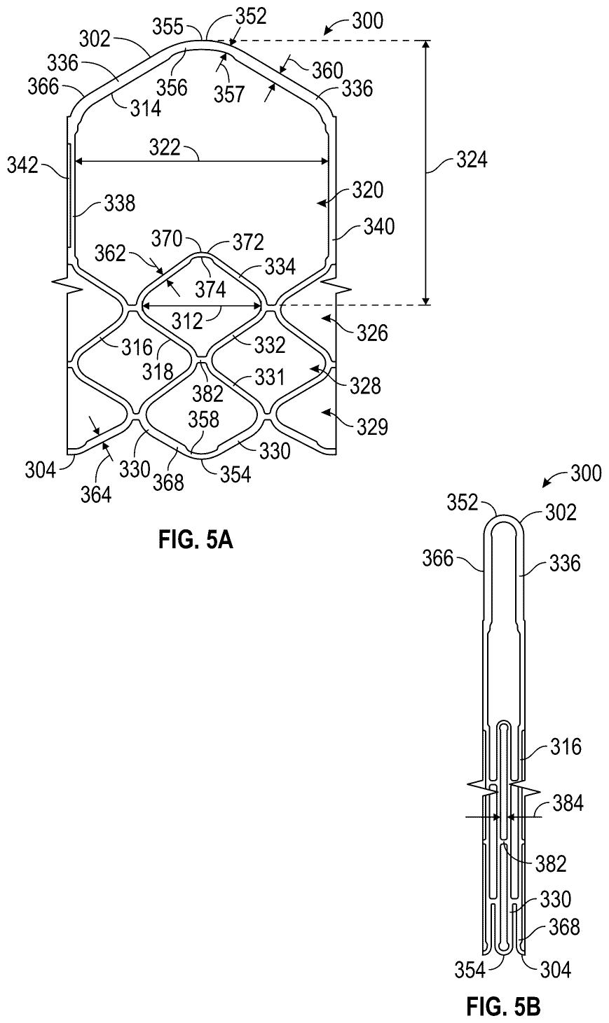

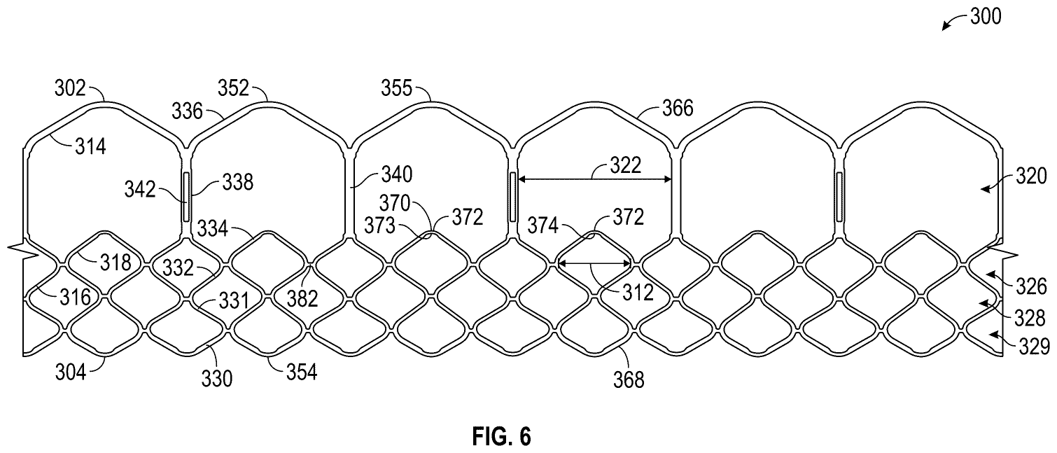

In some examples, the cells of the first row of cells can also be wider in a circumferential direction relative to cells of remaining rows of cells of the frame, as shown in FIGS. 5A-6. In some instances, each cell of the first row of cells can span a width of two cells of a second row of cells that is disposed adjacent to the first row of cells, thereby resulting in free apex regions at first ends of a portion of the cells of the second row of cells (FIGS. 5A and 6). The free apex regions can have a downstream-facing surface with a constant convex curvature extending between the angled struts or strut portions to which they are connected (FIGS. 5A-6). In some examples, the free apex regions can have a similar shape to the apex regions at the outflow end and/or the inflow end of the frame (for example, as shown in FIGS. 1-3 and 5A-6).

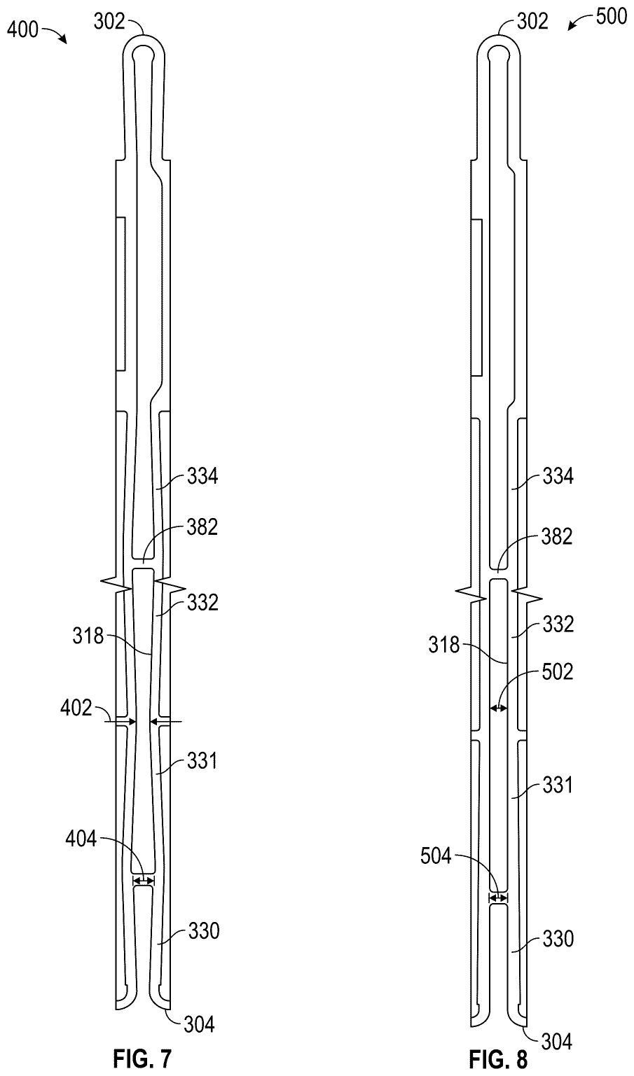

FIG. 5A shows a portion of the frame in a radially expanded state and FIG. 5B shows the portion of the frame in a radially compressed (or collapsed) state. In the radially compressed state, the angled struts of the frame can assume a more vertical orientation (extending in the axial direction) and be disposed closer to one another. The frame can further comprise relatively short horizontal struts extending between adjacent cells in a same row of cells which serve as spacers and are dimensioned to retain a minimal gap between adjacent struts in the radially compressed (crimped) state, thereby reducing a risk of pinching the leaflets (FIGS. 5A and 5B). The frame can be configured such that the angled struts assume a relatively straight vertical orientation (FIG. 8) or an inwardly bent shape (FIG. 7) in the compressed state. When being inwardly bent, the length of the horizontal struts can be selected to retain a minimal gap between the struts (at their narrowest point, for example).

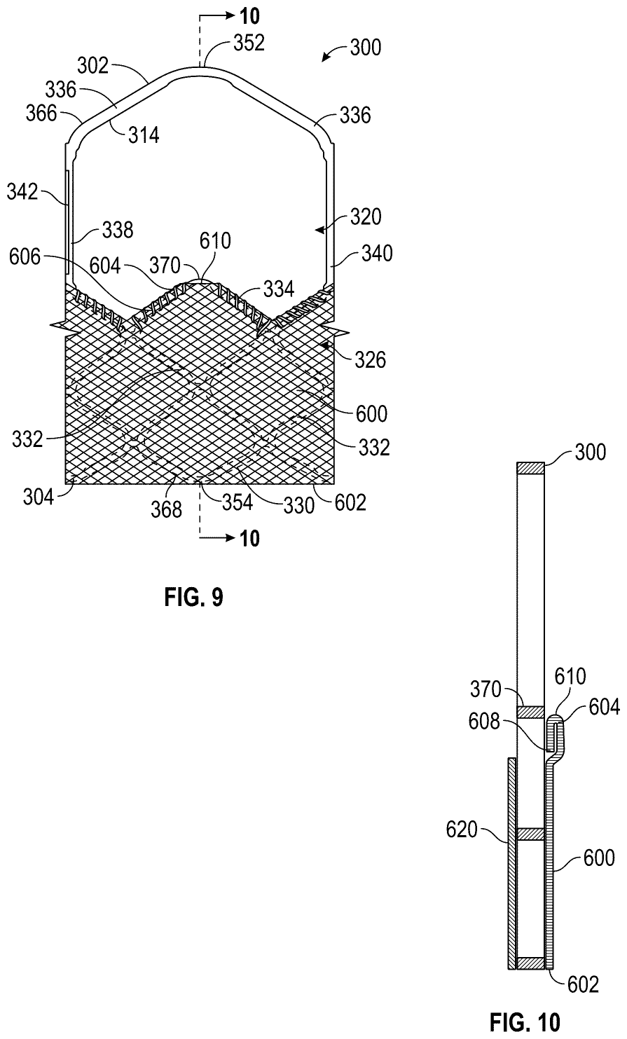

FIGS. 9 and 10 show an inner skirt disposed on an inner surface of the frame with its outflow edge arranged upstream of the free apex regions.

### Examples of the Disclosed Technology

FIG. 1 shows a prosthetic heart valve 100 (prosthetic valve), according to one example. Any of the prosthetic valves disclosed herein are adapted to be implanted in the native aortic annulus, although in other examples they can be adapted to be implanted in the other native annuluses of the heart (the pulmonary, mitral, and tricuspid valves). The disclosed prosthetic valves also can be implanted within vessels communicating with the heart, including a pulmonary artery (for replacing the function of a diseased pulmonary valve, or the superior vena cava or the inferior vena cava (for replacing the function of a diseased tricuspid valve) or various other veins, arteries and vessels of a patient. The disclosed prosthetic valves also can be implanted within a previously implanted prosthetic valve (which can be a prosthetic surgical valve or a prosthetic transcatheter heart valve) in a valve-in-valve procedure.

In some examples, the disclosed prosthetic valves can be implanted within a docking or anchoring device that is implanted within a native heart valve or a vessel. For example, in one example, the disclosed prosthetic valves can be implanted within a docking device implanted within the pulmonary artery for replacing the function of a diseased pulmonary valve, such as disclosed in U.S. Publication No. 2017/0231756, which is incorporated by reference herein. In another example, the disclosed prosthetic valves can be implanted within a docking device implanted within or at the native mitral valve, such as disclosed in PCT Publication No. WO2020/247907, which is incorporated herein by reference. In another example, the disclosed prosthetic valves can be implanted within a docking device implanted within the superior or inferior vena cava for replacing the function of a diseased tricuspid valve, such as disclosed in U.S. Publication No. 2019/0000615, which is incorporated herein by reference.

The prosthetic heart valve 100 can include a stent or frame 102, a valvular structure 104, and a perivalvular outer sealing member or outer skirt 106. The prosthetic heart valve 100 (and the frame 102) can have an inflow end 108 and an outflow end 110. The valvular structure 104 can be disposed on an interior of the frame 102 while the outer skirt 106 is disposed around an outer surface of the frame 102.

The valvular structure 104 can comprise a plurality of leaflets 112 (e.g., three leaflets, as shown in FIG. 1), collectively forming a leaflet structure, which can be arranged to collapse in a tricuspid arrangement. The leaflets 112 can be secured to one another at their adjacent sides (e.g., commissure tabs) to form commissures 114 of the valvular structure 104. For example, each leaflet 112 can comprise opposing commissure tabs disposed on opposite sides of the leaflet 112 and a cusp edge portion extending between the opposing commissure tabs. The cusp edge portion of the leaflets 112 can have an undulating, curved scalloped shape, and can be secured directly to the frame 102 (e.g., by sutures). However, in alternate examples, the cusp edge portion of the leaflets 112 can be secured to an inner skirt which is then secured to the frame 102. In some examples, the leaflets 112 can be formed of pericardial tissue (e.g., bovine pericardial tissue), biocompatible synthetic materials, or various other suitable natural or synthetic materials as known in the art and described in U.S. Pat. No. 6,730,118, which is incorporated by reference herein.

In some examples, the outer skirt 106 can be an annular skirt. In some instances, the outer skirt 106 can comprise one or more skirt portions that are connected together and/or individually connected to the frame 102. The outer skirt 106 can comprise a fabric or polymeric material, such as ePTFE, PTFE, PET, TPU, UHMWPE, PEEK, PE, etc. In some instances, instead of having a relatively straight upper edge portion, as shown in FIG. 1, the outer skirt 106 can have an undulating upper edge portion that extends along and is secured to the angled struts 134. Examples of such outer skirts, as well as various other outer skirts, that can be used with the frame 102 can be found in U.S. provisional patent application No. 63/366,599 filed Jun. 17, 2022, which is incorporated by reference herein.

The frame 102 can be radially compressible and expandable between a radially compressed (or collapsed) configuration and a radially expanded configuration (the expanded configuration is shown in FIG. 1). The frame 102 is shown alone in FIG. 2 and a portion of the frame 102 in a straightened (non-annular) configuration is shown in FIG. 3.

The frame 102 can be made of any of various suitable plastically-expandable materials (e.g., stainless steel, etc.) or self-expanding materials (e.g., Nitinol). When constructed of a plastically-expandable material, the frame 102 (and thus the valve 100) can be crimped to a radially compressed state on a delivery catheter and then expanded inside a patient by an inflatable balloon or equivalent expansion mechanism. When constructed of a self-expandable material, the frame 102 (and thus the valve 100) can be crimped to a radially compressed state and restrained in the compressed state by insertion into a sheath or equivalent mechanism of a delivery catheter. Once inside the body, the valve can be advanced from the delivery sheath, which allows the valve to expand to its functional size.

Suitable plastically-expandable materials that can be used to form the frames disclosed herein (e.g., the frame 102) include, metal alloys, polymers, or combinations thereof. Example metal alloys can comprise one or more of the following: nickel, cobalt, chromium, molybdenum, titanium, or other biocompatible metal. In some examples, the frame 102 can comprise stainless steel. In some examples, the frame 102 can comprise cobalt-chromium. In some examples, the frame 102 can comprise nickel-cobalt-chromium. In some examples, the frame 102 comprises a nickel-cobalt-chromium-molybdenum alloy, such as MP35N™ (tradename of SPS technologies), which is equivalent to UNS R30035 (covered by ASTM F562-02). MP35N™/UNS R30035 comprises 35% nickel, 35% cobalt, 20% chromium, and 10% molybdenum, by weight.

As shown in FIGS. 2 and 3, the frame 102 can comprise a plurality of interconnected struts 116 which form multiple rows of open cells 118 between the outflow end 110 and the inflow end 108 of the frame 102. In some examples, as shown in FIGS. 2 and 3, the frame 102 can comprise three rows of cells 118 with a first (upper in the orientation shown in FIGS. 2 and 3) row of cells 120 disposed at the outflow end 110. The first row of cells 120 comprises cells 118 that are elongated in an axial direction (relative to a central longitudinal axis 122 of the frame 102), as compared to cells 118 in the remaining rows of cells. For example, the cells 118 of the first row of cells 120 can have a longer axial length 124 (FIG. 3) than cells 118 in the remaining rows of cells, which can include a second row of cells 126 and a third row of cells 128, the third row of cells 128 disposed at the inflow end 108 and the second row of cells 126 disposed between the first row of cells 120 and the third row of cells 128.

In some examples, as shown in FIG. 2, each row of cells comprises nine cells 118. Thus, in such examples, the frame 102 can be referred to as a nine-cell frame.

In alternate examples, the frame 102 can comprise more than three rows of cells (e.g., four or five) and/or more or less than nine cells per row. In some examples, the cells 118 in the first row of cells 120 may not be elongated compared to cells 118 in the remaining rows of cells of the frame 102 (the second row of cells 126 and the third row of cells 128).

The interconnected struts 116 can include a plurality of angled struts 130, 132, 134, and 136 arranged in a plurality of rows of circumferentially extending rows of angled struts, with the rows being arrayed along the length of the frame 102 between the outflow end 110 and the inflow end 108. For example, the frame 102 can comprise a first row of angled struts 130 arranged end-to-end and extending circumferentially at the inflow end 108 of the frame; a second row of circumferentially extending, angled struts 132; a third row of circumferentially extending, angled struts 134; and a fourth row of circumferentially extending, angled struts 136 at the outflow end 110 of the frame 102. The fourth row of angled struts 136 can be connected to the third row of angled struts 134 by a plurality of axially extending window struts 138 (or window strut portions) and a plurality of axial (or axially extending) struts 140. The axially extending window struts 138 (which can also be referred to as axial struts that include a commissure window) define commissure windows (e.g., open windows) 142 that are spaced apart from one another around the frame 102, in a circumferential direction, and which are adapted to receive a pair of commissure tabs of a pair of adjacent leaflets 112 arranged into a commissure (e.g., commissure 114 shown in FIG. 1). In some examples, the commissure windows 142 and/or the axially extending window struts 138 defining the commissure windows 142 can be referred to herein as commissure features or commissure supports, each commissure feature or support configured to receive and/or be secured to a pair of commissure tabs of a pair of adjacent leaflets.

One or more (for example, two, as shown in FIGS. 2 and 3) axial struts 140 can be positioned between, in the circumferential direction, two commissure windows 142 formed by the window struts 138. Since the frame 102 can include fewer cells per row (e.g., nine) and fewer axial struts 140 between each commissure window 142, as compared to some more traditional prosthetic heart valves, each cell 118 can have an increased width (in the circumferential direction), thereby providing a larger opening for blood flow and/or coronary access.

Each axial strut 140 and each window strut 138 extends from a location defined by the convergence of the lower ends (e.g., ends arranged inward of and farthest away from the outflow end 110) of two angled struts 136 (which can also be referred to as an upper strut junction or upper elongated strut junction) to another location defined by the convergence of the upper ends (e.g., ends arranged closer to the outflow end 110) of two angled struts 134 (which can also be referred to as a lower strut junction or lower elongate strut junction). Each axial strut 140 and each window strut 138 forms an axial side of two adjacent cells of the first row of cells 120.

In some examples, as shown in FIG. 3, each axial strut 140 can have a width 144 (FIG. 3) that is larger than a width of the angled struts 130, 132, 134, and 136. As used herein, a “width” of a strut is measured between opposing locations on opposing surfaces of a strut that extend between the radially facing inner and outer surfaces of the strut (relative to the central longitudinal axis 122 of the frame 102). A “thickness” of a strut is measured between opposing locations on the radially facing inner and outer surfaces of a strut and is perpendicular to the width of the strut. In some examples, the width 144 of the axial struts 140 is 50-200%, 75-150%, or at least 100% larger than (e.g., double) the width of the angled struts of the frame 102.

By providing the axial struts 140 with the width 144 that is greater than the width of other, angled struts of the frame 102, a larger contact area is provided for when the leaflets 112 contact the wider axial struts 140 during systole, thereby distributing the stress and reducing the extent to which the leaflets 112 may fold over the axial struts 140, radially outward through the cells 118. As a result, a long-term durability of the leaflets 112 can be increased.

Since the cells 118 of the frame 102 can have a relatively large width compared to alternate prosthetic valves that have more than nine cells per row (as introduced above), the wider axial struts 140 can be more easily incorporated into the frame 102, without sacrificing open space for blood flow and/or coronary access.

Commissure tabs 115 of adjacent leaflets 112 can be secured together to form commissures 114 (FIG. 1). Each commissure 114 of the prosthetic heart valve 100 comprises two commissure tabs 115 paired together, one from each of two adjacent leaflets 112, and extending through a commissure window 142 of the frame 102. Each commissure 114 can be secured to the window struts 138 forming the commissure window 142.

The cusp edge portion (e.g., scallop edge) of each leaflet 112 can be secured to the frame 102 via one or more fasteners (e.g., sutures). In some examples, the cusp edge portion of each leaflet 112 can be secured directly to the struts of the frame 102 (e.g., angled struts 130, 132, and 134). For example, the cusp edge portions of the leaflets 112 can be sutured to the angled struts 130, 132, and 134 that generally follow the contour of the cusp edge portions of the leaflets 112.

In some examples, the cusp edge portion of the leaflets 112 can be secured to an inner skirt and the inner skirt can then be secured directly to the frame 102.

Various methods for securing the leaflets 112 to a frame, such as the frame 102, are disclosed in U.S. provisional patent applications 63/278,922, filed Nov. 12, 2021, and 63/300,302, filed Jan. 18, 2022, both of which are incorporated by reference herein.

As shown in FIGS. 2 and 3, in some examples, one or more of or each of the axial struts 140 can comprise an inflow end portion 146 (e.g., an end portion that is closest to the inflow end 108) and an outflow end portion 148 that are widened relative to a middle portion 150 of the axial strut 140 (which can be defined by the width 144). In some instances, the inflow end portion 146 of the axial strut 140 can comprise an aperture 147. The apertures 147 can be configured to receive fasteners (e.g., sutures) for attaching soft components of the prosthetic heart valve 100 to the frame 102. For example, in some instances, the outer skirt 106 can be positioned around the outer surface of the frame 102 and an upper or outflow edge portion of the outer skirt 106 can be secured to the apertures 147 by fasteners 149 (e.g., sutures), as shown in FIG. 1.

The interconnected struts 116 can also comprise horizontal struts 182 that extend between adjacent cells 118 of a row of cells of the frame 102 (FIGS. 2 and 3). The horizontal struts 182 can extend in a circumferential direction and also be referred to as circumferentially extending struts 182. The horizontal struts 182 can connect angled struts of two adjacent rows of angled struts of the frame 102 to one another. For example, each horizontal strut 182 can connect to two angled struts of one row of struts (for example, struts 134 shown in FIG. 3) and two angled struts in another, adjacent row of struts (for example, struts 132 shown in FIG. 3). As a result, an angled strut 184 extending between an axially extending window strut 138 and the horizontal strut 182 and an angled strut 186 extending between the horizontal strut 182 and another horizontal strut 182 disposed adjacent to the inflow end 108 of the frame 102 can be aligned along an angled line that can follow a scallop line of the leaflets (when the leaflets are attached to the frame 102). Thus, the horizontal struts 182 can allow the angled struts to follow a shape that more closely matches a shape of the scallop line of the leaflets when the frame 102 is in the radially expanded configuration (as shown in FIGS. 2 and 3). Additionally, the horizontal struts 182 can serve as spacers that can maintain a specified gap between the angled struts when the frame 102 is in the radially compressed configuration, thereby reducing a risk of pinching the leaflets between the struts in the radially compressed configuration.

The frame 102 can further comprise a plurality of apex regions 152 formed at the inflow end 108 and the outflow end 110, each apex region 152 extending and forming a junction between two angled struts 130 at the inflow end 108 or two angled struts 136 at the outflow end 110. As such, the apex regions 152 are spaced apart from one another, in a circumferential direction at the inflow end 108 and the outflow end 110.

Each apex region 152 can comprise an apex 154 (the highest or most outward extending, in an axial direction, point) and two thinned (or narrowed) strut portions 156, one thinned strut portion 156 extending from either side of the apex 154 to a corresponding, wider, angled strut 136 (at the outflow end 110) or angled strut 130 (at the inflow end 108) (FIG. 3). In this way, each of the apex regions 152 at the outflow end 110 can form a narrowed transition region between and relative to the two angled struts 136 extending from the corresponding apex region 152 and each of the apex regions 152 at the inflow end 108 can form a narrowed transition region between and relative to the two angled struts 130 extending from the corresponding apex region 152.

The thinned strut portions 156 of the apex regions 152 can have a width 158 that is smaller than a width 160 of the angled struts 130 or 136 (FIG. 3). In some examples, the width 158 can be a uniform width (e.g., along an entire length of the strut portion 156). In some examples, the width 158 of the thinned strut portions 156 can be from about 0.06-0.15 mm smaller than the width 160 of the angled struts 130 and/or 136.

The thinned strut portions 156 of the apex regions 152 can have a first length 162 (FIG. 3). In some examples, the first length 162 is in a range of 0.8-1.4 mm, 0.9-1.2 mm, 0.95-1.05 mm, or about 1.0 mm (e.g., +0.03 mm). In alternate examples, the first length 162 is in a range of 0.3-0.7 mm, 0.4-0.6 mm, 0.45-0.55 mm, or about 0.5 mm (e.g., +0.03 mm).

Thus, each outflow apex region 152 can include two thinned strut portions 156 having the first length 162, each extending from the apex 154, outward relative to a central longitudinal axis 164 of the cells 118. Thus, a total length of the apex region 152 can be two times the first length 162.

Each apex region 152 and two corresponding angled struts 136 at the outflow end 110 can form an outflow strut 166 and each apex region 152 and two corresponding angled struts 130 at the inflow end 108 can form an inflow strut 168.

Each outflow strut 166 and inflow strut 168 can have a length that includes an apex region 152 and the two angled struts 136 or 130 (or strut portions), respectively, on either side of the apex region 152. One half the total length of each outflow strut 166 and inflow strut 168 is shown in FIG. 3 as length 170, which extends from an end of one angled strut 136 or 130 to the central longitudinal axis 164. Thus, the length of each outflow strut 166 and inflow strut 168 is two times length 170. In some examples, the length 170 for half of each inflow strut 168 can be different than the length 170 for half of each outflow strut 166.

In some instances, the length of each thinned strut portion 156 can be at least 25% of the length 170 of the corresponding half outflow strut 166 or inflow strut 168. Said another way, the length of each apex region 152 (a total length being two times the first length 162) can be at least 25% of the total length (two times length 170) of the outflow strut 166 or inflow strut 168. In some examples, the length of each apex region 152 can be more than 25% of the total length of the corresponding outflow strut 166 or inflow strut 168, such as 25-35%.

In some examples, each apex region 152 can comprise a curved, axially facing outer surface 172 and an arcuate or curved, axially facing inner depression 174 which forms the thinned strut portions 156. For example, the curved inner depression 174 can depress toward the curved outer surface 172 from an inner surface of the angled strut portions 156, thereby forming the smaller width thinned strut portions 156. Thus, the curved inner depressions 174 can be formed on a cell side of the apex region 152 (e.g., as opposed to the outside of the apex region 152).

In some examples, the curved outer surface 172 of each apex region 152 can form a single, continuous curve from one angled strut portion 156 on a first side of the apex region 152 to another angled strut portion 156 on an opposite, second side of the apex region 152 (for example, the curved outer surface 172 can have a constant convex curvature).

As used herein, “constant convex curvature” can refer to a continuously curved surface which is convex and which does not have an inflection point (no change in direction of the curvature).

Each apex region 152 can have a radius of curvature 176, along the curved outer surface 172 (e.g., in some instances, along an entirety or an entire length of the curved outer surface 172) (FIG. 3). In some instances, the radius of curvature 176 at the apex 154 and/or along the entire curved outer surface 172 of the apex region 152 can be greater than 1 mm. In some instances, the radius of curvature 176 can be in a range of 1-20 mm, 3-16 mm, or 8-14 mm. In some instances, the radius of curvature 176 can be greater than 10 mm. The radius of curvature 176 can be dependent on (and thus change due to changes in) the width 158 (e.g., the amount of reduction in width from the angled struts 130 or 136) and the first length 162 of the thinned strut portions 156.

Further, a height (an axial height) 178 of the apex regions 152, which can be defined in the axial direction from an outer surface of the two angled struts 130 or 136 to the curved outer surface 172 of the apex region 152 at the apex 414, can be the width 158 of the thinned strut portions 156 (FIG. 3). In this way, the height 178 of the apex regions 152 can be relatively small and not add much to the overall axial height of the radially expanded frame 102. Thus, the leaflets 112 secured to the frame 102 (FIG. 1) can be disposed close to the inflow end 108, thereby leaving a lar If ger open space at the outflow end 110 of the frame 102 that is not blocked by the leaflets 112.

In some examples, each of the apex region 152 can form an angle 180 between the two angled struts 130 or 136 extending from either side of the corresponding apex region 152 (FIG. 3). In some instances, the angle 180 can be in a range of 120 (not inclusive) to 140 degrees (e.g., such that the angle 180 is greater than 120 degrees and less than or equal to 140 degrees).

Additional details and examples of frames for prosthetic heart valves that include apex regions can be found in PCT Application No. PCT/US2022/025687, which is incorporated by reference herein.

FIG. 4 shows a delivery apparatus 200, according to an example, that can be used to implant an expandable prosthetic heart valve (e.g., the prosthetic heart valve 100 of FIG. 1 and/or any of the other prosthetic heart valves described herein). In some examples, the delivery apparatus 200 is specifically adapted for use in introducing a prosthetic valve into a heart.

The delivery apparatus 200 in the illustrated example of FIG. 4 is a balloon catheter comprising a handle 202 and a steerable, outer shaft 204 extending distally from the handle 202. The delivery apparatus 200 can further comprise an intermediate shaft 206 (which also may be referred to as a balloon shaft) that extends proximally from the handle 202 and distally from the handle 202, the portion extending distally from the handle 202 also extending coaxially through the outer shaft 204. Additionally, the delivery apparatus 200 can further comprise an inner shaft 208 extending distally from the handle 202 coaxially through the intermediate shaft 206 and the outer shaft 204 and proximally from the handle 202 coaxially through the intermediate shaft 206.

The outer shaft 204 and the intermediate shaft 206 can be configured to translate (e.g., move) longitudinally, along a central longitudinal axis 220 of the delivery apparatus 200, relative to one another to facilitate delivery and positioning of a prosthetic valve at an implantation site in a patient's body.

The intermediate shaft 206 can include a proximal end portion 210 that extends proximally from a proximal end of the handle 202, to an adaptor 212. A rotatable knob 214 can be mounted on the proximal end portion 210 and can be configured to rotate the intermediate shaft 206 around the central longitudinal axis 220 and relative to the outer shaft 204.

The adaptor 212 can include a first port 238 configured to receive a guidewire therethrough and a second port 240 configured to receive fluid (e.g., inflation fluid) from a fluid source. The second port 240 can be fluidly coupled to an inner lumen of the intermediate shaft 206.

The intermediate shaft 206 can further include a distal end portion that extends distally beyond a distal end of the outer shaft 204 when a distal end of the outer shaft 204 is positioned away from an inflatable balloon 218 of the delivery apparatus 200. A distal end portion of the inner shaft 208 can extend distally beyond the distal end portion of the intermediate shaft 206.

The balloon 218 can be coupled to the distal end portion of the intermediate shaft 206.

In some examples, a distal end of the balloon 218 can be coupled to a distal end of the delivery apparatus 200, such as to a nose cone 222 (as shown in FIG. 4), or to an alternate component at the distal end of the delivery apparatus 200 (e.g., a distal shoulder). An intermediate portion of the balloon 218 can overlay a valve mounting portion 224 of a distal end portion of the delivery apparatus 200 and a distal end portion of the balloon 218 can overly a distal shoulder 226 of the delivery apparatus 200. The valve mounting portion 224 and the intermediate portion of the balloon 218 can be configured to receive a prosthetic heart valve in a radially compressed state. For example, as shown schematically in FIG. 4, a prosthetic heart valve 250 (which can be one of the prosthetic valves described herein) can be mounted around the balloon 218, at the valve mounting portion 224 of the delivery apparatus 200.

The balloon shoulder assembly, including the distal shoulder 226, is configured to maintain the prosthetic heart valve 250 (or other medical device) at a fixed position on the balloon 218 during delivery through the patient's vasculature.

The outer shaft 204 can include a distal tip portion 228 mounted on its distal end. The outer shaft 204 and the intermediate shaft 206 can be translated axially relative to one another to position the distal tip portion 228 adjacent to a proximal end of the valve mounting portion 224, when the prosthetic valve 250 is mounted in the radially compressed state on the valve mounting portion 224 (as shown in FIG. 4) and during delivery of the prosthetic valve to the target implantation site. As such, the distal tip portion 228 can be configured to resist movement of the prosthetic valve 250 relative to the balloon 218 proximally, in the axial direction, relative to the balloon 218, when the distal tip portion 228 is arranged adjacent to a proximal side of the valve mounting portion 224.

An annular space can be defined between an outer surface of the inner shaft 208 and an inner surface of the intermediate shaft 206 and can be configured to receive fluid from a fluid source via the second port 240 of the adaptor 212. The annular space can be fluidly coupled to a fluid passageway formed between the outer surface of the distal end portion of the inner shaft 208 and an inner surface of the balloon 218. As such, fluid from the fluid source can flow to the fluid passageway from the annular space to inflate the balloon 218 and radially expand and deploy the prosthetic valve 250.

An inner lumen of the inner shaft can be configured to receive a guidewire therethrough, for navigating the distal end portion of the delivery apparatus 200 to the target implantation site.

The handle 202 can include a steering mechanism configured to adjust the curvature of the distal end portion of the delivery apparatus 200. In the illustrated example, for example, the handle 202 includes an adjustment member, such as the illustrated rotatable knob 260, which in turn is operatively coupled to the proximal end portion of a pull wire. The pull wire can extend distally from the handle 202 through the outer shaft 204 and has a distal end portion affixed to the outer shaft 204 at or near the distal end of the outer shaft 204. Rotating the knob 260 can increase or decrease the tension in the pull wire, thereby adjusting the curvature of the distal end portion of the delivery apparatus 200. Further details on steering or flex mechanisms for the delivery apparatus can be found in U.S. Pat. No. 9,339,384, which is incorporated by reference herein.

The handle 202 can further include an adjustment mechanism 261 including an adjustment member, such as the illustrated rotatable knob 262, and an associated locking mechanism including another adjustment member, configured as a rotatable knob 278. The adjustment mechanism 261 is configured to adjust the axial position of the intermediate shaft 206 relative to the outer shaft 204 (e.g., for fine positioning at the implantation site). Further details on the delivery apparatus 200 can be found in PCT Application No. PCT/US2021/047056, which is incorporated by reference herein.

FIGS. 5A-6 illustrate another exemplary frame 300 for a prosthetic heart valve. In some examples, the frame 300 can be used in lieu of the frame 102 in the prosthetic heart valve 100 of FIG. 1. The frame 300 can be radially compressible and expandable between a radially compressed (or collapsed) configuration (FIG. 5B) and a radially expanded configuration (FIG. 5A). A portion of the frame 300 is shown in FIGS. 5A and 5B while an entirety of the frame 300 is shown in a straightened (non-annular) configuration in FIG. 8. It should be noted that the frame 300 can assume an annular configuration, such as that shown in FIG. 2.

The frame 300 can comprise any of the plastically-expandable materials (e.g., stainless steel, etc.) or self-expanding materials (e.g., nickel titanium alloy (NiTi), such as nitinol) materials discussed above with reference to the frame 102.

The frame 300 can comprise a plurality of interconnected struts 316 which form multiple rows of open cells 314, 318 between an outflow end 302 and an inflow end 304 of the frame 300. In some examples, as shown in FIGS. 5A-6, the frame 300 can comprise four rows of cells 314, 318 with a first (upper in the orientation shown in FIGS. 5A-6) row of cells 320 disposed at the outflow end 302. The first row of cells 320 comprises cells 314 that are elongated in an axial direction (relative to a central longitudinal axis of the frame 300 and a direction extending between the outflow end 302 and the inflow end 304) and wider in a circumferential direction, as compared to cells 318 in the remaining rows of cells. For example, the cells 314 of the first row of cells 320 can have a longer axial length 324 (FIG. 5A, as measured from an outflow end to an inflow end of the cell 320) and a larger width 322 than cells 318 in the remaining rows of cells. The increased width 322 and axial length 324 of the cells 314 of the first row can provide a larger opening for coronary access through the frame 300.

The remaining rows of cells can include a second row of cells 326, a third row of cells 328, and a fourth row of cells 329. The fourth row of cells 329 is disposed at the inflow end 304 and the second row of cells 326 is disposed between the first row of cells 320 and the third row of cells 328 (which is disposed adjacent to the fourth row of cells 329).

In some examples, as shown in FIG. 6, each of the second row of cells 326, the third row of cells 328, and the fourth row of cells 329 comprise 12 cells 318 and the first row of cells 320 comprises six cells 314. This arrangement of differing numbers of cells between the first row of cells 320 and the remaining rows of cells is due to the width 322 of the cells 314 of the first row of cells 320 being twice as wide as a width 312 of the cells 318 of the remaining rows of cells (for example, the adjacent second row of cells 326). Thus, in some examples, each cell 314 of the first row of cells 320 can span the width of two cells 314 (such as the third row of cells 328, as depicted in FIGS. 5A and 6). Said another way, the width 322 of each cell 314 can be double the width 312 of a cell 318.

In alternate examples, the frame 300 can comprise a different number of rows of cells, such as three (for example, similar to the frame 102 of FIGS. 2 and 3) or five. Additionally or alternatively, in some instances the frame 300 can include more or less than 12 and six cells per row (for example, ten and five cells per row). In some alternate examples, the cells 314 in the first row of cells 320 may not be elongated compared to cells 318 in the remaining rows of cells of the frame 300. In still other alternate examples, the width 322 of the cells 314 can be larger or smaller than shown in FIGS. 5A and 6 (such as being 1.5 times wider than the width 312 of the cells 318).

The interconnected struts 316 can include a plurality of angled struts 330, 331, 332, 334, and 336 arranged in a plurality of rows of circumferentially extending rows of angled struts, with the rows being arrayed along the length of the frame 300 between the outflow end 302 and the inflow end 304. For example, the frame 300 can comprise a first row of angled struts 330 arranged end-to-end and extending circumferentially at the inflow end 304 of the frame; a second row of circumferentially extending, angled struts 331; a third row of circumferentially extending, angled struts 332; a fourth row of circumferentially extending, angled struts 334, and a fifth row of circumferentially extending, angled struts 336 at the outflow end 302 of the frame 300. The circumferentially extending rows of angled struts (as well as additional components, such as the rows of cells) can be referred to herein as being “upstream” or “downstream” of other circumferentially extending rows of angled struts. As used herein, “upstream” or “downstream” are relative to the outflow end 302 of the frame (which is the downstream end of the frame) and the inflow end 304 of the frame (which is the upstream end of the frame) and a direction of blood flow through the frame 300 (from the inflow end 304 to the outflow end 302). For example, the fourth row of angled struts 334 is disposed upstream of the fifth row of angled struts 336.

The fifth row of angled struts 336 can be connected to the fourth row of angled struts 334 by a plurality of axially extending window struts 338 (which can be configured similar to the window struts 138 of frame 102, as described above) and a plurality of axial (or axially extending) struts 340 (which can be configured similar to the axial struts 140 of frame 102, as described above). The axially extending window struts 338 (which can also be referred to as axial struts that include a commissure window) define commissure windows (e.g., open windows) 342 that are spaced apart from one another around the frame 300, in a circumferential direction, and which are adapted to receive a pair of commissure tabs of a pair of adjacent leaflets arranged into a commissure (for example, commissure 114 shown in FIG. 1). In some examples, the commissure windows 342 and/or the axially extending window struts 338 defining the commissure windows 342 can be referred to herein as commissure features or commissure supports, each commissure feature or support configured to receive and/or be secured to a pair of commissure tabs of a pair of adjacent leaflets.

As shown in FIG. 6, one axial strut 340 can be positioned between, in the circumferential direction, two commissure windows 342 formed by the window struts 338. Each axial strut 340 and each window strut 338 extends from a location defined by the convergence of the lower ends (e.g., ends arranged inward of and farthest away from the outflow end 302) of two angled struts 336 (which can also be referred to as an upper strut junction or upper elongated strut junction) to another location defined by the convergence of the upper ends (e.g., ends arranged closer to the outflow end 302) of two angled struts 334 (which can also be referred to as a lower strut junction or lower elongate strut junction). Each axial strut 340 and each window strut 338 forms an axial side of two adjacent cells of the first row of cells 320.

In some examples, a width 360 of the angled struts 336 can be larger than a width of the struts of the remaining rows of struts (for example, to provide increased strength to the larger/wider cells 314). For example, the width 360 can be larger than a width 362 of the angled struts 334 (which, in some examples, can also be the width of the angled struts 331 and 332). Additionally, in some examples, a width 364 of the angled struts 330 can be larger than the width 362. However, in some examples, the width 360 of the angled struts 336 can still be larger than the width 364 of the angled struts 330.

In some examples, the frame 300 can further comprise a plurality of apex regions 352 formed at the outflow end 302 and plurality of apex regions 354 formed at the inflow end 304. Each apex region 354 can extend and form a junction between two angled struts 330 at the inflow end 304 of the frame 300. The apex regions 354 can be configured the same as or similarly to the apex regions 152, as described above (and thus are not redescribed here for the sake of brevity).

Each apex region 352 can extend and form a junction between two angled struts 336 at the outflow end 302 of the frame 300. The apex regions 352 can also be configured similarly to the apex regions 152, as described above. For example, each apex region 352 can comprise an apex 355 and two thinned (or narrowed) strut portions 356 (FIG. 5A) extending from either side of the apex 355 to a corresponding, wider, angled strut 336 (at the outflow end 302). The thinned strut portions 356 of the apex regions 352 can have a width 357 that is smaller than a width 360 of the angled struts 336 (FIG. 5A).

In some examples, the width 357 can be from about 0.06-0.15 mm smaller than the width 360 of the angled struts 336. For example, in some instances, the width 360 can be approximately 0.46 mm and the width 357 can be approximately 0.4 mm.

However, since the cells 314 are wider as compared to cells in remaining rows of the frame 300 (and as compared to the cells 118 of the frame 102), the angled struts 336 have a greater length than the angled struts 330. Thus, the length of thinned strut portions 356 of each apex region 352 can be longer than the thinned strut portions 358 of each apex region 354, for example (FIG. 5A). However, relative proportions between the thinned strut portion 356 of the apex regions 352 and the angled struts 336 can be similar or the same as those described above for the apex regions 152 of frame 102 (for example, a length of the thinned strut portions 356 can be at least 25% of the length of the angled strut 336)

Each apex region 352 and two corresponding angled struts 336 at the outflow end 302 can form an outflow strut 366 and each apex region 354 and two corresponding angled struts 330 at the inflow end 304 can form an inflow strut 368. Due to the increased width 322 of the cells 314 of the first row of cells 320, the outflow struts 366 are longer than the inflow struts 368.

A length of the apex region 352 (e.g., an arc length along the two thinned strut portions 356, similar to the length 162 shown in FIG. 3) is at least 25% of a length of the outflow strut 366. Similarly, a length of the apex region 354 is at least 25% of a length of the inflow strut 368.

An overall shape or curvature of the apex regions 352 and 354 can be similar to that of apex regions 152, as described above. Exemplary dimensions for the apex regions 352 and 354 are described below in reference to the free apex regions 370.

In this way, the apex regions 352 can have a relatively small axial height and not add much to the overall axial height of the radially expanded frame 300 (similar to as described above for the apex regions 152, and despite the longer length of the outflow struts 366). As such, the axial height of the radially expanded frame 300 can be smaller than alternate valve frames that have apices that are more pointed and/or have a larger axial height.

Due to the increased width 322 of the cells 314 of the first row of cells 320 relative to the width 312 of the cells 318 of the second row of cells 326, a portion of the second row of cells 320 can be formed by struts with exposed or free apex regions 370 that are unattached to additional struts of the adjacent row of cells (the first row of cells 320). As used herein, “free apex regions” can refer to apex regions that are not attached (directly attached) to any other struts, except for the angled struts which they curve and extend between (such as a pair of angled struts 334 and a corresponding apex region 370 disposed therebetween).

For example, the free apex regions 370 are not attached to any axial struts 340 or axially extending window struts 338. As shown in FIGS. 5A-6, every other cell 318 of the second row of cells 326 can be defined by struts having a free apex region 370. In alternate examples, more than one cell 318 defined by struts having a free apex region 370 can be disposed between cells 318 that are directly connected to an axial strut of the adjacent row of struts (for example, the axial struts 340 or axially extending window struts 338).

As introduced above, in some examples, the free apex regions 370 can interact with the leaflets of the prosthetic heart valve. For example, when the leaflets (such as the leaflets 112 of FIG. 1) are secured to the frame 300 as described herein, the free apex regions 370 can be disposed at a level (or axial height) of a portion of the leaflets that open and close during operation of the prosthetic heart valve. Thus, in some instances, when the leaflets are in an open state when implanted in a patient, the leaflets can contact the free apex regions 370.

Thus, it is beneficial for the free apex regions 370 to have a curved outer surface 372, similar to the apex regions 352 and 354. For example, as shown in FIGS. 5A-6, each free apex region 370 can have a similar overall shape to the apex regions 352 and 354, as described above, such as having a continuously curved outer surface 372 with a constant convex curvature that curves between the angled struts 334 to which it is connected (for example, with a radius of curvature in any of the ranges described herein for any of the curved apex regions. The outer surface 372 can face in a downstream direction, or toward the outflow end 302.

As used herein, “constant convex curvature” can refer to a continuously curved surface which is convex and which does not have an inflection point (no change in direction of the curvature).

Each free apex region 370 can extend and form a junction between two angled struts 334 at and can comprise an apex and two thinned (or narrowed) strut portions extending from either side of the apex to a corresponding, wider, angled strut 334 (similar to the apex regions 352, 354 and/or 152). An overall shape or curvature of the free apex regions 370 can be similar to that of apex regions 152, as described above.

The free apex regions 370 (e.g., the thinned strut portions and apex of the free apex regions 370) have a narrowed width relative to the angled struts 334 to which they are connected, thereby forming an inner depression 374 on a cell side of each free apex region 370 (for example, the inner depression 374 is formed on an opposing inner surface 373 of the free apex region 370 that faces an upstream direction, or toward the inflow end 304). The width of the free apex regions 370 can be defined between the outer surface 372 and the inner surface 373.

The width of the free apex regions 370 can be from about 0.06-0.15 mm smaller than the width of the angled struts 334. For example, in some instances, the width of the free apex regions 370 can be approximately 0.18 mm and the width of the angled struts 334 can be approximately 0.24 mm.

A length of each free apex region 370 (e.g., an arc length along the apex regions 370, similar to the length 162 shown in FIG. 3) is at least 25% a length of the entire strut it is part of, where the strut is defined the include the free apex region 370 and the two angled struts 335 to which it is connected.

In some instances, the length of the free apex regions 370, the outflow apex regions 352, and/or the inflow apex regions 354 can be in a range of 0.5-4.7 mm or 0.9-3.7 mm (e.g., with the free apex regions 370 being on the lower end of the range and the outflow apex regions 352 being on the higher end of the range).

In some examples, a radius of curvature of the free apex regions 370, the outflow apex regions 352, and/or the inflow apex regions 354 can be in a range of 0.3-10 mm, 0.5-8 mm, or 0.2-20 mm.

In some instances, a ratio between the width of any of the apex regions 370, 352, and 354 and a width of the angled struts to which they connect (e.g., a ratio between the width of the free apex regions 370 and the width of the angled struts 334 and/or a ratio between the width 357 and the width 360 of the apex regions 352) can be in a range of 0.15-0.98, 0.4-0.8, or 0.6-0.9.

In some examples, a central longitudinal axis of each free apex region 370 can be aligned (overlapping) with a central longitudinal axis of a corresponding apex region 352. In alternate examples, the free apex regions 370 may not be aligned with corresponding apex regions 352 (for example, if the cells 314 are not twice as wide as the cells 318 in the second row of cells 326).

In this way, the free apex regions 370 can have a larger curved (less angled) outer surface that is more atraumatic and may not interfere with the leaflets of the prosthetic heart valve as the leaflets open and close during operation of the prosthetic heart valve. Thus, a long term durability of the prosthetic valve leaflets can be increased.

In alternative examples, the free apex regions 370 of the frame 300 may not include the curved outer surface 372 with the constant convex curvature. Instead, the free apex regions 370 can have an alternative shape that is similar to the junction between two angled struts 334 that connect to an axial strut 340 or axially extending window struts 338.

As shown in FIGS. 5A-6, the interconnected struts 316 of the frame 300 can also comprise horizontal struts 382 (which can be similar to horizontal struts 182 of frame 102, as described above) that extend between adjacent cells 318 of a row of cells of the frame 300. The horizontal struts 382 can connect angled struts of two adjacent rows of angled struts of the frame 300 to one another. As described above with reference to the horizontal struts 182 of the frame 102, the horizontal struts 382 can allow the angled struts 330, 331, 332, and 334 to follow a shape that closely matches a shape of the scallop line of the leaflets of the prosthetic valve when the frame 300 is in the radially expanded configuration (FIG. 5A). Additionally, the horizontal struts 382 can serve as spacers that maintain a specified gap 384 between the angled struts 330, 331, 332, and 334 when the frame 300 is in the radially compressed configuration (FIG. 5B), where the specified gap 384 can be minimized as much as possible (to minimize a crimp profile of the prosthetic valve) while still being large enough to reduce a risk of catching or pinching the leaflets of the prosthetic valve between the adjacent angled struts 330, 331, 332, and 334 when the frame 300 is radially compressed (or crimped).

FIGS. 7 and 8 illustrate two examples of prosthetic valve frames 400 and 500, respectively, including horizontal struts 382 and angled struts 330, 331, 332, and 334 that assume either a relatively straight vertical orientation (FIG. 8) or an inwardly bent orientation (FIG. 7) when the frame is radially compressed (or crimped). The frames 400 and 500 of FIGS. 7 and 8, respectively, can be similar to the frame 300, but without the wider outflow cells (cells 314). However, it should be noted that the angling or straight orientation of the angled struts 330, 331, 332, and 334, as described below with reference to FIGS. 7 and 8, can also be applied to the frame 300.

When the angled struts 330, 331, 332, and 334 assume the inwardly bent (or angled) orientation in the compressed state of the frame 400 (FIG. 7), a length 404 of the horizontal struts 382 can be specified to retain a minimal gap 402 between the angled struts, as measured as the smallest width value (in the circumferential direction of the frame 400) between adjacent horizontal struts 382, for example. The minimal gap 402 can reduce a crimp profile of the frame 400 while avoiding pinching leaflets between the angled struts in the crimped state.

When the angled struts 330, 331, 332, and 334 assume the relatively straight vertical orientation (in the axial direction) in the compressed state of the frame 500 (FIG. 8), a length 504 of the horizontal struts 382 can be specified to retain a minimal gap 502 between the angled struts. The minimal gap 502 can reduce a crimp profile of the frame 500 while avoiding pinching leaflets between the angled struts in the crimped state.

In some examples, the horizontal struts 382 are dimensioned to retain the minimal gap 402 or 502 within a range of about 0.2 mm-0.7 mm or about 0.3 mm-0.4 mm.

In other examples, the minimal gap 402 or 502 in the crimped configuration can be a function of the prosthetic valve leaflet's thickness, and can account for compressibility of the tissue of the leaflets. For example, for a leaflet having a thickness of about 0.2 mm., a minimal gap 402 or 502 can be designed to accommodate a leaflet folded over itself, which may require a gap of at least 0.4 mm. However, if the tissue has about 50% compressibility, it may be sufficient to design for a minimal gap of about 0.2 mm.

In this way, a frame of a prosthetic heart valve can be configured to increase a durability and long term longevity of the prosthetic heart valve (for example, when implanted), while also reducing a crimp profile of the prosthetic heart valve and reducing stresses on the frame struts (for example, due to a shape of the apex regions).

As introduced above, in some examples, the prosthetic valve can comprise an inner skirt disposed on an inner surface of the frame (any one of the frames described here or similar frames). In some examples, cusp edge portions of the leaflets (e.g., leaflets 112) can be secured to the inner skirt and the inner skirt can then be secured directly to the frame.

FIGS. 9 and 10 show an exemplary inner skirt 600 disposed on an inner surface of the frame 300. For example, FIG. 9 shows an interior view of the frame 300 with the inner skirt 600 arranged against inner surfaces of the angled struts 330, 331, 332, 334, and FIG. 10 shows a cross-sectional view of the frame 300 and inner skirt 600 of FIG. 9 with an outer skirt 620 disposed around an outer surface of the frame 300. Although the inner skirt 600 is depicted on the frame 300, it should be noted that the inner skirt 600 can be arranged similarly on different prosthetic valve frames which have free apices or apex regions.

The inner skirt 600 can have an inflow edge portion 602 disposed at the inflow end 304 of the frame 300, and an outflow edge portion 604. The outflow edge portion 604 can have a zig-zag shape. For example, as shown in FIG. 9, the outflow edge portion 604 can comprise a plurality of peaks spaced apart from one another in a circumferential direction. The outflow edge portion 604 can also comprise a plurality of valleys spaced apart from one another in the circumferential direction, with one valley disposed between two adjacent peaks. In some examples, a shape of the outflow edge portion 604 can follow a shape of the fourth row of angled struts 334 (or second row of struts relative to the outflow end 302). The outflow edge portion 604 can be secured to the angled struts 334 with a plurality of stitches 606, for example.

At each free apex region 370 (or free apex in another frame), a respective peak of the outflow edge portion 604 of the inner skirt 600 can be aligned with the free apex region 370. At least one peak can be arranged below or upstream of the respective apex region 370, thereby exposing the free apex region 370 (as shown in FIGS. 9 and 10). For example, the outflow edge portion 604 does not cover an entirety of the inner surface of the free apex region 370, and as a result, the apex region 370 protrudes above (or downstream of) the outflow edge portion 604.

In some examples, the peaks of the outflow edge portion 604 can comprise a trimmed, flat, or straight edge 610 at each apex region 370. The straight edges 610 of the inner skirt 600 can be disposed upstream of and away from the respective apex regions 370. Said another way, the straight edges 610 can be spaced from the respective apex regions 370 toward an inflow end of the frame.

In some examples, the outflow edge portion 604 (or portions thereof) is folded outward, toward the frame 300, and over itself (as shown in FIG. 10). As a result, an outflow edge 608 of the outflow edge portion 604 is disposed between (sandwiched between) an inner surface of the frame 300 and an adjacent portion of the inner skirt 600 (as shown in FIG. 10). Thus, in some examples, one or more peaks of the outflow edge portion 604 can be folded to form a fold line that forms the respective straight edge 610.

In some examples, the peaks of the outflow edge portion 604 include a plurality of first peaks that are aligned with respective apices of the angled struts 334 that connect to axially extending window struts 338, and a plurality of second peaks that are aligned with respective free apex regions 370. As noted above, the second peaks can have straight edges 610, whereas the first peaks can be pointed (e.g., not trimmed or folded over). As such, in some examples, the first peaks can extend axially toward an outflow end of the frame by a greater extent than the second peaks.

In some examples, the outflow edge 608 is molten or rougher that a remainder of the inner skirt 600. Thus, folding the outflow edge portion 604 over in this way can hide the outflow edge 608 away from an interior of the prosthetic valve, thereby keeping the leaflets from contacting the outflow edge 608.

When the frame 300 (or another frame to which the inner skirt 600 is attached) is radially compressed (or crimped) into a radially compressed configuration, the outflow edge portion 604 of the inner skirt 600 slides upstream on the frame 300, and farther away from the free apex regions 370. During radial expansion of the frame (e.g., to a radially expanded configuration), the outflow edge portion 604 is pulled up by the stitches 606 sliding on the angled struts 334, but not all the way to the apex regions 370.

This ensures that the flat or straight edges 610 of the inner skirt 600 remain at or below (upstream of) the level of the free apex regions 370, thereby reducing the likelihood of the outflow edge 608 contacted the tissue of the leaflets. Further, by including the inner skirt 600 on the frame 300 including the curved apex regions 370 (as described above), the longevity of the leaflets can be further increased.

The inner skirt 600 can be wholly or partly formed of any suitable biological material, synthetic material (for example, any of various polymers), or combinations thereof. In some examples, the inner skirt 600 can comprise a fabric having interlaced yarns or fibers, such as in the form of a woven, braided, or knitted fabric. In some examples, the fabric can have a plush nap or pile. Exemplary fabrics having a plus nap or pile include velour, velvet, velveteen, corduroy, terrycloth, fleece, etc. In some examples, the inner skirt 600 can comprise a fabric without interlaced yarns or fibers or randomly interlaced yarns or fibers, such as felt or an electrospun fabric. Exemplary materials that can be used for forming such fabrics (with or without interlaced yarns or fibers) include, without limitation, polyethylene (PET), ultra-high molecular weight polyethylene (UHMWPE), polytetrafluoroethylene (PTFE), expanded polytetrafluoroethylene (ePTFE), polyamide etc. In some examples, the inner skirt 600 can comprise a non-textile or non-fabric material, such as a film made from any of a variety of polymeric materials, such as PTFE, PET, polypropylene, polyamide, polyetheretherketone (PEEK), polyurethane (such as thermoplastic polyurethane (TPU), etc. In some examples, the inner skirt 600 can comprise a sponge material or foam, such as polyurethane foam. In some examples, the inner skirt 600 can comprise natural tissue, such as pericardium (for example, bovine pericardium, porcine pericardium, equine pericardium, or pericardium from other sources).

### Delivery Techniques

For implanting a prosthetic valve within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded (e.g., by inflating a balloon, actuating one or more actuators of the delivery apparatus, or deploying the prosthetic valve from a sheath to allow the prosthetic valve to self-expand). Alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve (on the distal end portion of the delivery apparatus) is introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire previously inserted into a patient's vasculature. Moreover, the disclosed delivery approaches are not intended to be limited. Any of the prosthetic valves disclosed herein can be implanted using any of various delivery procedures and delivery devices known in the art.

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A prosthetic heart valve comprising: a radially expandable and compressible annular frame comprising a plurality of interconnected angled struts defining a plurality of circumferentially extending rows of cells arranged between an outflow end and an inflow end of the frame, wherein the plurality of interconnected angled struts is arranged to form a plurality of circumferentially extending rows of struts, including a first row of struts at the outflow end of the frame, a second row of struts upstream of the first row of struts, and a third row of struts upstream of the second row of struts, wherein the plurality of circumferentially extending rows of cells comprises: a first row of first cells disposed at the outflow end and at least partially defined by the first and second rows of struts; and a second row of second cells disposed upstream of the first row of first cells and at least partially defined by the second and third rows of struts, wherein each first cell of the first row of first cells has a first width that is larger than a second width of each second cell of the second row of second cells, and wherein the second row of struts comprises a plurality of free apex regions, and wherein each free apex region connects adjacent ends of a respective pair of angled struts of the second row of struts together and has a first surface facing in a downstream direction and an opposing second surface facing in an upstream direction, wherein the first surface has a constant convex curvature extending between the adjacent ends of the respective pair of angled struts.

Example 2. The prosthetic heart valve of any example herein, particularly example 1, wherein the second surface of each free apex region defines a depressed region between the adjacent ends of the respective pair of angled struts such that a width of the free apex region between the first and second surfaces is less than a width of the respective pair of angled struts.

Example 3. The prosthetic heart valve of any example herein, particularly either example 1 or example 2, wherein the first surface of the free apex region forms a single, continuous convex curve from a downstream facing surface of a first angled strut of the respective pair of angled struts that is disposed on a first side of the free apex region to a downstream facing surface of a second angled strut of the respective pair of angled struts that is disposed on an opposite, second side of the free apex region.

Example 4. The prosthetic heart valve of any example herein, particularly any one of examples 1-3, wherein the first width is two times wider than the second width.

Example 5. The prosthetic heart valve of any example herein, particularly any one of examples 1-4, wherein the first row of first cells includes six cells and the second row of second cells includes twelve cells.

Example 6. The prosthetic heart valve of any example herein, particularly any one of examples 1-5, wherein the frame further comprises a plurality of axially extending struts that extend between the first row of struts and the second row of struts and define axial sides of the first row of first cells.

Example 7. The prosthetic heart valve of any example herein, particularly example 6, wherein a first portion of struts of the second row of struts form pairs of angled struts that are each connected to a respective axially extending strut of the plurality of axially extending struts, wherein a second portion of struts of the second row of struts form pairs of angled struts that are connected by a respective free apex region of the plurality of apex regions, and wherein the plurality of free apex regions are unattached to the plurality of axially extending struts.

Example 8. The prosthetic heart valve of any example herein, particularly either example 6 or example 7, wherein a portion of the plurality of axially extending struts are axially extending window struts that define commissure windows, further comprising a plurality of leaflets secured together at their adjacent sides to form commissures, and wherein the commissures are secured to the commissure windows of the frame.

Example 9. The prosthetic heart valve of any example herein, particularly any one of examples 1-8, wherein the first row of struts form pairs of angled struts, wherein struts of each pair of angled struts of the first row are connected together at their adjacent ends by an outflow apex region, and wherein the outflow apex region curves between the pair of angled struts and has a narrowed width relative to the pair of angled struts.

Example 10. The prosthetic heart valve of any example herein, particularly example 9, wherein the outflow apex region has a third surface and an opposing fourth surface facing in the upstream direction, and wherein the third surface has a constant convex curvature extending between the pair of angled struts.

Example 11. The prosthetic heart valve of any example herein, particularly any one of examples 1-10, wherein each first cell of the first row of first cells has a first axial length that is longer than a second axial length of each second cell of the second row of second cells.

Example 12. The prosthetic heart valve of any example herein, particularly any one of examples 1-11, wherein the frame further comprises a plurality of horizontal struts that extend between adjacent second cells in the second row of second cells, and wherein each horizontal strut of the plurality of horizontal struts connects two adjacent struts of the second row of struts to two adjacent struts of the third row of struts.

Example 13. The prosthetic heart valve of any example herein, particularly example 12, wherein a length, in a circumferential direction, of each horizontal strut is specified to maintain a specified gap between the two adjacent struts of the second row of struts and the two adjacent struts of the third row of struts when the frame is in a radially compressed configuration.

Example 14. The prosthetic heart valve of any example herein, particularly example 13, wherein in the radially compressed configuration, the second row of struts and the third row of struts are oriented axially, in a relatively straight vertical orientation relative to a central longitudinal axis of the frame.

Example 15. The prosthetic heart valve of any example herein, particularly example 13, wherein in the radially compressed configuration, the second row of struts and the third row of struts extend axially, but angle inward toward one another at adjacent horizontal struts.

Example 16. The prosthetic heart valve of any example herein, particularly any one of examples 1-15, wherein each strut in the first row of struts has a first width that is larger than a second width of each strut in the second row of struts.

Example 17. The prosthetic heart valve of any example herein, particularly example 16, wherein the plurality of circumferentially extending rows of struts further includes a fourth row of struts at the inflow end of the frame, and wherein each strut of the fourth row of struts has a third width that is smaller than the first width and larger than the second width.

Example 18. The prosthetic heart valve of any example herein, particularly any one of examples 1-17, further comprising a plurality of leaflets secured on an inside of the frame and configured to open and close in order to regulate a flow of blood through the prosthetic heart valve, from the inflow end to the outflow end of the frame, wherein each free apex region of the second row of struts is disposed at a level of a portion of the plurality of leaflets that open and close during operation of the prosthetic heart valve.

Example 19. A prosthetic heart valve comprising: a radially expandable and collapsible annular frame comprising a plurality of interconnected struts defining a plurality of circumferentially extending rows of cells arranged between an outflow end and an inflow end of the frame, wherein the plurality of interconnected struts comprises: a plurality of circumferentially extending rows of angled struts including a first row of struts at the outflow end of the frame, a second row of struts upstream of the first row of struts, and a third row of struts upstream of the second row of struts; and a plurality of axial struts spaced circumferentially apart around the frame and extending between the first row of struts and the second row of struts, wherein the plurality of circumferentially extending rows of cells comprises: a first row of first cells disposed at the outflow end and at least partially defined by the first and second rows of struts and the plurality of axial struts; and a second row of second cells disposed upstream of the first row of first cells and at least partially defined by the second and third rows of struts, wherein each first cell of the first row of first cells has a first width that is larger than a second width of each second cell of the second row of second cells, and wherein the second row of second cells comprises: a first portion of second cells that are defined by first pairs of angled struts of the second row of struts that are directly connected to the plurality of axial struts at their adjacent ends; and a second portion of second cells that are defined by second pairs of angled struts of the second row of struts and a plurality of free apex regions, wherein each free apex region connects adjacent ends of a respective second pair of angled struts together and has a first surface facing in a downstream direction and an opposing second surface facing in an upstream direction, wherein the first surface has a constant convex curvature extending between the adjacent ends of the respective second pair of angled struts, and wherein the plurality of free apex regions are unattached to the plurality of axial struts.

Example 20. The prosthetic heart valve of any example herein, particularly example 19, wherein the second surface of each free apex region defines an inner depression that depresses inward from upstream facing surfaces of the respective second pair of angled struts toward the first surface of the apex region such that a width of the apex region between its first and second surfaces is smaller than a width of the respective second pair of angled struts.

Example 21. The prosthetic heart valve of any example herein, particularly either example 19 or example 20, wherein each strut of the first row of struts comprises two angled strut portions interconnected by an outflow apex region, and wherein the outflow apex region curves between the two angled strut portions, with a constant convex curvature at its downstream-facing surface, and has a narrowed with relative to the two angled strut portions.

Example 22. The prosthetic heart valve of any example herein, particularly example 21, wherein each outflow apex region forms an angle between the two angled strut portions that is greater than 120 degrees.

Example 23. The prosthetic heart valve of any example herein, particularly either example 21 or example 22, wherein each outflow apex region is aligned with and spaced axially apart from a corresponding free apex region.

Example 24. The prosthetic heart valve of any example herein, particularly any one of examples 19-23, wherein the first width is two times wider than the second width.

Example 25. The prosthetic heart valve of any example herein, particularly any one of examples 19-24, wherein each first cell of the first row of first cells has a first axial length that is longer than a second axial length of each second cell of the second row of second cells.

Example 26. The prosthetic heart valve of any example herein, particularly any one of examples 19-25, further comprising a plurality of leaflets secured on an inside of the frame and configured to open and close in order to regulate a flow of blood through the prosthetic heart valve, from the inflow end to the outflow end of the frame, wherein each free apex region of the second row of struts is disposed at a level of a portion of the plurality of leaflets that open and close during operation of the prosthetic heart valve.

Example 27. The prosthetic heart valve of any example herein, particularly example 26, wherein the frame is radially expandable and collapsible between a radially expanded configuration and a radially compressed configuration, and wherein the frame further comprises a plurality of horizontal struts that extend between adjacent second cells in the second row of second cells, wherein each horizontal strut of the plurality of horizontal struts connects two adjacent struts of the second row of struts to two adjacent struts of the third row of struts and is configured to maintain a specified gap between the two adjacent struts of the second row of struts and the two adjacent struts of the third row of struts when the frame is in a radially collapsed configuration.

Example 28. The prosthetic heart valve of any example herein, particularly any one of examples 19-27, wherein each strut in the first row of struts has a first width that is larger than a second width of each strut in the second row of struts.

Example 29. The prosthetic heart valve of any example herein, particularly example 28, wherein the plurality of circumferentially extending rows of angled struts further includes a fourth row of struts at the inflow end of the frame, and wherein each strut of the fourth row of struts has a third width that is smaller than the first width.

Example 30. A prosthetic heart valve comprising: a radially expandable and collapsible annular frame comprising a plurality of interconnected struts defining a plurality of circumferentially extending rows of cells arranged between an outflow end and an inflow end of the frame, wherein the plurality of interconnected struts comprises: a circumferentially extending row of first struts defining the outflow end, each first strut comprising two angled strut portions interconnected by an outflow apex region, wherein the outflow apex region curves between the two angled strut portions and has a narrowed width relative to a width of the two angled struts portions; a plurality of axially extending struts spaced circumferentially apart around the frame and connected to the row of first struts; a circumferentially extending row of angled second struts disposed upstream of the row of first struts, wherein a first portion of second struts of the row of angled second struts are each directly connected to a respective axially extending strut of the plurality of axially extending struts, and wherein a second portion of second struts of the row of angled second struts form pairs of second struts which are connected together by a free apex region that is unattached to the plurality of axially extending struts, wherein the free apex region of each respective pair of second struts has a first surface facing in a downstream direction and an opposing second surface facing in an upstream direction, wherein the first surface curves between the respective pair of second struts, and wherein the second surface depresses inward toward the first surface such that a width of the free apex region between the first and second surfaces is less than a width of the respective pair of second struts; and a circumferentially extending row of angled third struts, wherein the row of first struts, the axially extending struts, and the row of angled second struts form a first row of cells of the plurality of rows of cells disposed at the outflow end, wherein the row of angled second struts and the row of angled third struts form a second row of cells of the plurality of rows of cells that are disposed adjacent to the first row of cells, and wherein a first width of each cell of the first row of cells is wider than a second width of each cell of the second row of cells; and a plurality of leaflets secured on an inside of the frame and configured to open and close in order to regulate a flow of blood through the prosthetic heart valve, from the inflow end to the outflow end of the frame, wherein each free apex region of the row of angled second struts is disposed at a level of a portion of the plurality of leaflets that open and close during operation of the prosthetic heart valve.

Example 31. The prosthetic heart valve of any example herein, particularly example 30, wherein the first surface of each free apex region has a constant convex curvature between downstream facing surfaces of the respective pair of second struts.

Example 32. The prosthetic heart valve of any example herein, particularly either example 30 or example 31, wherein the outflow apex region of each first strut forms an angle between the two angled strut portions that is greater than 120 degrees.

Example 33. The prosthetic heart valve of any example herein, particularly any one of examples 30-32, wherein a first surface of the outflow apex region of each first strut that faces away from the inflow end of the frame forms a single, continuous curve with a convex curvature from one angled strut portion of the two angled strut portions on a first side of the outflow apex region to another angled strut portion of the two angled strut portions on a second side of the outflow apex region.

Example 34. The prosthetic heart valve of any example herein, particularly any one of examples 30-33, wherein the first width is two times wider than the second width.

Example 35. The prosthetic heart valve of any example herein, particularly any one of examples 30-34, wherein each cell of the first row of cells has a first axial length that is longer than a second axial length of each cell of the second row of cells.

Example 36. The prosthetic heart valve of any example herein, particularly any one of examples 30-35, wherein the frame is radially expandable and collapsible between a radially expanded configuration and a radially collapsed configuration, and wherein the frame further comprises a plurality of horizontal struts that extend between adjacent cells of the second row of cells, wherein each horizontal strut of the plurality of horizontal struts connects together two adjacent second struts of the row of angled second struts and two adjacent third struts of the row of angled third struts and is configured to maintain a specified gap, in a circumferential direction, between the two adjacent second struts and the two adjacent third struts when the frame is in a radially collapsed configuration.

Example 37. The prosthetic heart valve of any example herein, particularly any one of examples 30-36, wherein the width of the two angled strut portions of each first strut in the row of first struts is larger than a width of each second strut in the row of angled second struts.

Example 38. The prosthetic heart valve of any example herein, particularly example 37, wherein the plurality of interconnected struts further comprises a circumferentially extending row of angled fourth struts at the inflow end of the frame, and wherein each fourth strut of the row of angled fourth struts has a width that is smaller than the width of the two angled strut portion of each first strut.

Example 39. An assembly comprising: a delivery apparatus comprising a balloon; and an implantable prosthetic heart valve that is radially collapsible to a collapsed configuration and radially expandable to an expanded configuration, the prosthetic heart valve comprising: a radially expandable and compressible annular frame comprising a plurality of interconnected angled struts defining a plurality of circumferentially extending rows of cells arranged between an outflow end and an inflow end of the frame, wherein the plurality of interconnected angled struts is arranged to form a plurality of circumferentially extending rows of struts, including a first row of struts at the outflow end of the frame, a second row of struts upstream of the first row of struts, and a third row of struts upstream of the second row of struts, wherein the plurality of circumferentially extending rows of cells comprises: a first row of first cells disposed at the outflow end and at least partially defined by the first and second rows of struts; and a second row of second cells disposed upstream of the first row of first cells and at least partially defined by the second and third rows of struts, wherein each first cell of the first row of first cells has a first width that is larger than a second width of each second cell of the second row of second cells, and wherein the second row of struts comprises a plurality of free apex regions, and wherein each free apex region connects together a respective pair of angled struts of the second row of struts and has a first surface facing the outflow end of the frame and an opposing second surface facing the inflow end of the frame, wherein the first surface forms a single, continuous convex curve from one angled strut of the respective pair of angled struts on a first side of the free apex region to another angled strut of the respective pair of angled struts on an opposite, second side of the free apex region, wherein the collapsed prosthetic heart valve can be mounted around the balloon and radially expanded to the expanded configuration with the balloon inside a patient's body.

Example 40. The assembly of any example herein, particularly example 39, wherein each free apex region has a width, between the first and second surfaces of the free apex region, that is smaller than a width of the respective pair of angled struts.

Example 41. The assembly of any example herein, particularly either example 39 or example 40, wherein the second surface of each free apex region defines a depression between the first and second sides of the free apex region that depresses toward the first surface such that a width of the free apex region between the first and second surfaces is less than a width of the respective pair of angled struts.

Example 42. The assembly of any example herein, particularly any one of examples 39-41, wherein the first width is double the second width.

Example 43. The assembly of any example herein, particularly any one of examples 39-41, wherein the first row of first cells includes half the number of cells included in the second row of second cells.

Example 44. The assembly of any example herein, particularly any one of examples 39-43, wherein the frame further comprises a plurality of axially extending struts that extend between the first row of struts and the second row of struts and define axial sides of the first row of first cells.

Example 45. The assembly of any example herein, particularly example 44, wherein a first portion of struts of the second row of struts form pairs of angled struts that are each connected to a respective axially extending strut of the plurality of axially extending struts, wherein a second portion of struts of the second row of struts form pairs of angled struts that are connected by a respective free apex region of the plurality of apex regions, and wherein the plurality of free apex regions are unattached to the plurality of axially extending struts.

Example 46. The assembly of any example herein, particularly either example 44 or example 45, further comprising a plurality of leaflets, each leaflet comprising opposing commissure tabs disposed on opposite sides of the leaflet and a cusp edge portion extending between the opposing commissure tabs, wherein a portion of the plurality of axially extending struts are axially extending window struts that define commissure windows, and wherein commissure tabs of adjacent leaflets are paired together and secured to a respective commissure window of the frame.

Example 47. The assembly of any example herein, particularly any one of examples 39-46, wherein the first row of struts form pairs of angled struts, wherein the struts of each pair of angled struts are connected together at their adjacent ends by an outflow apex region, and wherein the outflow apex region curves between the pair of angled struts and has a narrowed width relative to the pair of angled struts.

Example 48. The assembly of any example herein, particularly example 47, wherein the outflow apex region has a third surface and an opposing fourth surface facing the inflow end of the frame, and wherein the third surface has a constant convex curvature extending between the pair of angled struts.

Example 49. The assembly of any example herein, particularly any one of examples 39-48, wherein each first cell of the first row of first cells has a first axial length that is longer than a second axial length of each second cell of the second row of second cells.

Example 50. The assembly of any example herein, particularly any one of examples 39-49, wherein the frame further comprises a plurality of horizontal struts that extend between adjacent second cells of the second row of second cells, and wherein each horizontal strut of the plurality of horizontal struts connects two adjacent struts of the second row of struts to two adjacent struts of the third row of struts.

Example 51. The assembly of any example herein, particularly example 50, wherein a length, in a circumferential direction, of each horizontal strut is specified to maintain a specified gap between the two adjacent struts of the second row of struts and the two adjacent struts of the third row of struts when the frame is in the collapsed configuration, and wherein in the collapsed configuration the struts of the first, second, and third row of struts assume a more axially extending configuration than when the frame is in the expanded configuration.

Example 52. The assembly of any example herein, particularly any one of examples 39-51, wherein each strut in the first row of struts has a first width that is larger than a second width of each strut in the second row of struts.

Example 53. The assembly of any example herein, particularly example 52, wherein the plurality of circumferentially extending rows of struts further includes a fourth row of struts at the inflow end of the frame, and wherein each strut of the fourth row of struts has a third width that is smaller than the first width and larger than the second width.

Example 54. The assembly of any example herein, particularly any one of examples 39-53, wherein the prosthetic heart valve further comprises a plurality of leaflets secured on an inside of the frame and configured to open and close in order to regulate a flow of blood through the prosthetic heart valve, from the inflow end to the outflow end of the frame, wherein each free apex region of the second row of struts is disposed at a level of a portion of the plurality of leaflets that open and close during operation of the prosthetic heart valve.

Example 55. A prosthetic heart valve comprising: a radially expandable and compressible annular frame comprising a plurality of interconnected angled struts defining a plurality of circumferentially extending rows of cells arranged between a first end and a second end of the frame, wherein the plurality of interconnected angled struts is arranged to form a plurality of circumferentially extending rows of struts, including a first row of struts at the first end of the frame, a second row of struts disposed adjacent to the first row of struts, and a third row of struts disposed adjacent to the second row of struts, the second row of struts disposed between the first and third row of struts, wherein the plurality of circumferentially extending rows of cells comprises: a first row of first cells disposed at the first end and at least partially defined by the first and second rows of struts; and a second row of second cells disposed adjacent to the first row of first cells and at least partially defined by the second and third rows of struts, wherein each first cell of the first row of first cells has a first width that is larger than a second width of each second cell of the second row of second cells, and wherein the second row of struts comprises a plurality of free apex regions, and wherein each free apex region connects adjacent ends of a respective pair of angled struts of the second row of struts together and has a first surface facing the first end of the frame and an opposing second surface facing the second end of the frame, wherein the first surface has a constant convex curvature extending between the adjacent ends of the respective pair of angled struts.

Example 56. The prosthetic heart valve of any example herein, particularly example 55, wherein the second surface of each free apex region defines a depressed region between the adjacent ends of the respective pair of angled struts such that a width of the free apex region between the first and second surfaces is less than a width of the respective pair of angled struts.

Example 57. The prosthetic heart valve of any example herein, particularly either example 55 or example 56, wherein the first surface of the free apex region forms a single, continuous curve from a surface of a first angled strut of the respective pair of angled struts that faces the first end of the frame and is disposed on a first side of the free apex region to a surface of a second angled strut of the respective pair of angled struts that faces the second end of the frame and is disposed on an opposite, second side of the free apex region.

Example 58. The prosthetic heart valve of any example herein, particularly any one of examples 55-57, wherein the first width is two times wider than the second width.

Example 59. The prosthetic heart valve of any example herein, particularly any one of examples 55-58, wherein the first row of first cells includes six cells and the second row of second cells includes twelve cells.

Example 60. The prosthetic heart valve of any example herein, particularly any one of examples 55-59, wherein the frame further comprises a plurality of axially extending struts that extend between the first row of struts and the second row of struts and define axial sides of the first row of first cells.

Example 61. The prosthetic heart valve of any example herein, particularly example 60, wherein a first portion of struts of the second row of struts form pairs of angled struts that are each connected to a respective axially extending strut of the plurality of axially extending struts, wherein a second portion of struts of the second row of struts form pairs of angled struts that are connected by a respective free apex region of the plurality of apex regions, and wherein the plurality of free apex regions are unattached to the plurality of axially extending struts.

Example 62. The prosthetic heart valve of any example herein, particularly either example 60 or example 61, wherein a portion of the plurality of axially extending struts are axially extending window struts that define commissure windows, further comprising a plurality of leaflets secured together at their adjacent sides to form commissures, and wherein the commissures are secured to the commissure windows of the frame.

Example 63. The prosthetic heart valve of any example herein, particularly any one of examples 55-62, wherein the first row of struts form pairs of angled struts, wherein the struts of each pair of angled struts are connected together at their adjacent ends by an outflow apex region, and wherein the outflow apex region curves between the pair of angled struts and has a narrowed width relative to the pair of angled struts.

Example 64. The prosthetic heart valve of any example herein, particularly example 63, wherein the outflow apex region has a third surface and an opposing fourth surface facing in the second end of the frame, and wherein the third surface has a constant convex curvature extending between the pair of angled struts.

Example 65. The prosthetic heart valve of any example herein, particularly any one of examples 55-64, wherein each first cell of the first row of first cells has a first axial length that is longer than a second axial length of each second cell of the second row of second cells.

Example 66. The prosthetic heart valve of any example herein, particularly any one of examples 55-65, wherein the frame further comprises a plurality of horizontal struts that extend between adjacent second cells in the second row of second cells, and wherein each horizontal strut of the plurality of horizontal struts connects two adjacent struts of the second row of struts to two adjacent struts of the third row of struts.

Example 67. The prosthetic heart valve of any example herein, particularly example 66, wherein a length, in a circumferential direction, of each horizontal strut is specified to maintain a specified gap between the two adjacent struts of the second row of struts and the two adjacent struts of the third row of struts when the frame is in a radially compressed configuration.

Example 68. The prosthetic heart valve of any example herein, particularly example 67, wherein in the radially compressed configuration, the second row of struts and the third row of struts are oriented axially, in a relatively straight vertical orientation relative to a central longitudinal axis of the frame.

Example 69. The prosthetic heart valve of any example herein, particularly example 67, wherein in the radially compressed configuration, the second row of struts and the third row of struts extend axially, but angle inward toward one another at adjacent horizontal struts.

Example 70. The prosthetic heart valve of any example herein, particularly any one of examples 55-69, wherein each strut in the first row of struts has a first width that is larger than a second width of each strut in the second row of struts.

Example 71. The prosthetic heart valve of any example herein, particularly example 70, wherein the plurality of circumferentially extending rows of struts further includes a fourth row of struts at the second end of the frame, and wherein each strut of the fourth row of struts has a third width that is smaller than the first width and larger than the second width.

Example 72. The prosthetic heart valve of any example herein, particularly any one of examples 55-71, further comprising a plurality of leaflets secured on an inside of the frame and configured to open and close in order to regulate a flow of blood through the prosthetic heart valve, from the second end to the first end of the frame, wherein each free apex region of the second row of struts is disposed at a level of a portion of the plurality of leaflets that open and close during operation of the prosthetic heart valve.

Example 73. The prosthetic heart valve of any example herein, particularly any one of examples 55-72, wherein the first end is an outflow end of the frame and the second end is an inflow end of the frame.

Example 74. A prosthetic heart valve comprising: a radially expandable and compressible annular frame comprising a plurality of interconnected struts defining a plurality of circumferentially extending rows of cells arranged between an inflow end and an outflow end of the frame, the plurality of interconnected struts comprising: a circumferentially extending row of outflow struts defining the outflow end, wherein each outflow strut comprises two angled strut portions interconnected by an apex region, wherein each apex region curves between a corresponding pair of two angled strut portions and has a narrowed width and a length that extends along at least 25% of a total length of the outflow strut, wherein the narrowed width is smaller than a width of the two angled strut portions; and a circumferentially extending row of angled first struts disposed upstream of the row of outflow struts, wherein the row of outflow struts and the row of angled first struts at least partially form a first row of cells of the plurality of circumferentially extending rows of cells disposed at the outflow end, and wherein each cell of the first row of cells has a first width that is larger than a second width of cells of remaining rows of cells of the plurality of circumferentially extending rows of cells.

Example 75. The prosthetic heart valve of any example herein, particularly example 74, wherein the first width is two times larger than the second width.

Example 76. The prosthetic heart valve of any example herein, particularly either example 74 or example 75, wherein the plurality of interconnected struts further comprises a circumferentially extending row of angled second struts disposed upstream of the row of angled first struts, and wherein the row of angled first struts and the row of angled second struts form a second row of cells of the plurality of rows of cells that is disposed adjacent to and upstream of the first row of cells, and wherein each cell of the second row of cells has the second width which is half the first width.

Example 77. The prosthetic heart valve of any example herein, particularly example 76, wherein a portion of first struts of the row of angled first struts form pairs of first struts which are connected together by a free apex region that is unattached to additional struts forming the first row of cells, wherein the free apex region of each respective pair of second struts has a first surface facing in a downstream direction and an opposing second surface facing in an upstream direction, wherein the first surface curves between the respective pair of second struts, and wherein the second surface depresses inward toward the first surface such that a width of the free apex region between the first and second surfaces is less than a width of the respective pair of second struts.

Example 78. The prosthetic heart valve of any example herein, particularly any one of examples 74-77, wherein the plurality of interconnected struts further comprises a plurality of axially extending struts that extend between the row of outflow struts and the row of angled first struts and define axial sides of the first row of cells.

Example 79. The prosthetic heart valve of any example herein, particularly any one of examples 74-78, wherein each cell of the first row of cells has a longer axial length than cells of the remaining rows of cells.

Example 80. The prosthetic heart valve of any example herein, particularly any one of examples 74-79, further comprising a plurality of leaflets secured on an inside of the frame and configured to open and close in order to regulate a flow of blood through the prosthetic heart valve, from the inflow end to the outflow end of the frame.

Example 81. The prosthetic heart valve of any example herein, particularly any one of examples 74-80, wherein the narrowed width of each apex region is from 0.06 mm to 0.15 mm smaller than the width of the two angled strut portions.

Example 82. The prosthetic heart valve of any example herein, particularly any one of examples 1-81, further comprising an inner skirt disposed around an inner surface of the frame, wherein an outflow edge portion of the inner skirt is secured to the second row of struts, and wherein at each free apex region, the outflow edge portion is disposed upstream of the free apex region.

Example 83. The prosthetic heart valve of any example herein, particularly example 82, wherein the outflow edge portion is folded over itself such that an outflow edge of the inner skirt is disposed between the inner surface of the frame and an adjacent portion of the inner skirt.

Example 84. The prosthetic heart valve of any example herein, particularly either example 82 or example 83, wherein the outflow edge portion of the inner skirt is secured to the second row of struts with a plurality of stitches.

Example 85. The prosthetic heart valve of any example herein, particularly any one of examples 82-84, wherein each apex region is uncovered by the inner skirt.

Example 86. A prosthetic heart valve comprising: a radially expandable and compressible annular frame comprising a plurality of interconnected angled struts defining a plurality of circumferentially extending rows of cells arranged between a first end and a second end of the frame, wherein the plurality of interconnected angled struts is arranged to form a plurality of circumferentially extending rows of struts, including a first row of struts at the first end of the frame, a second row of struts disposed adjacent to the first row of struts, and a third row of struts disposed adjacent to the second row of struts, the second row of struts disposed between the first and third row of struts, wherein the plurality of circumferentially extending rows of cells comprises: a first row of first cells disposed at the first end and at least partially defined by the first and second rows of struts; and a second row of second cells disposed adjacent to the first row of first cells and at least partially defined by the second and third rows of struts, wherein each first cell of the first row of first cells has a first width that is larger than a second width of each second cell of the second row of second cells, and wherein the second row of struts comprises a plurality of free apices, and wherein each free apex connects adjacent ends of a respective pair of angled struts of the second row of struts together; and an inner skirt disposed around an inner surface of the frame, wherein a first edge portion of the inner skirt is secured to the second row of struts, wherein at each free apex the first edge portion is disposed away from the free apex toward the second end of the frame, and wherein a second edge portion of the inner skirt is disposed at the second end of the frame.

Example 87. The prosthetic heart valve of any example herein, particularly example 86, wherein the first edge portion is folded over itself such that an outer, first edge of the inner skirt is disposed between the inner surface of the frame and an adjacent portion of the inner skirt.

Example 88. The prosthetic heart valve of any example herein, particularly either example 86 or example 87, wherein the first edge portion of the inner skirt is secured to the second row of struts with a plurality of stitches.

Example 89. The prosthetic heart valve of any example herein, particularly any one of examples 86-88, wherein each free apex is uncovered by the inner skirt.

Example 90. The prosthetic heart valve of any example herein, particularly any one of examples 86-89, wherein the first end of the frame is an outflow end and the second end of the frame is an inflow end, wherein the first edge portion of the inner skirt is an outflow edge portion, and wherein at each free apex, the outflow edge portion of the inner skirt is disposed upstream of the free apex.

Example 91. The prosthetic heart valve of any example herein, particularly any one of examples 86-90, wherein each free apex has a first surface facing the first end of the frame and an opposing second surface facing the second end of the frame, and wherein the first surface has a constant convex curvature extending between the adjacent ends of the respective pair of angled struts.

Example 92. A prosthetic heart valve comprising: a radially expandable and compressible annular frame comprising a plurality of interconnected angled struts arranged to form a plurality of circumferentially extending rows of struts, including a first row of struts, a second row of struts downstream of the first row of struts, and a third row of struts downstream of the second row of struts; and an inner skirt disposed around an inner surface of the frame, wherein the skirt comprises an inflow edge and an outflow edge, wherein the outflow edge is stitched to the struts of the second row of struts and comprises a plurality of peaks spaced apart from each other in a circumferential direction, wherein the peaks are aligned with respective apices of the second row of struts, and wherein at least one peak has a straight edge that is spaced from the respective apex toward an inflow end of the frame.

Example 93. The prosthetic heart valve of any example herein, particularly example 92, wherein the at least one peak is folded to form a fold line that forms the straight edge.

Example 94. The prosthetic heart valve of any example herein, particularly any one of examples 92 or 93, wherein a first set of apices of the second row of struts are connected to the third row of struts by axially extending struts and a second set of apices of the second row of struts are free apices that are free of any axially extending struts connected to the third row of struts, wherein the outflow edge of the skirt comprises a plurality of first peaks aligned with apices of the first set of apices and a plurality of second peaks having a straight edge aligned with respective free apices of the second row of struts.

Example 95. The prosthetic heart of any example herein, particularly example 94, wherein each second peak is disposed between two adjacent first peaks.

Example 96. The prosthetic heart valve of any example herein, particularly any one of examples 94-95, wherein the first peaks are pointed.

Example 97. The prosthetic heart valve of any example herein, particularly any one of examples 94-96, wherein the first peaks extend axially toward an outflow end of the frame a greater extent than the second peaks.

Example 98. The prosthetic heart valve of any example herein, particularly any one of examples 94-97, wherein the second peaks are folded, and the first peaks are not folded.

Example 99. The prosthetic heart valve of any example herein, particularly any one of examples 94-98, wherein each free apex of the free apices connects adjacent ends of a respective pair of struts of the second row of struts, and wherein each free apex has a first surface facing in a downstream direction and an opposing second surface facing in an upstream direction, wherein the first surface has a constant convex curvature extending between the adjacent ends of the respective pair of struts.

Example 100. The prosthetic heart valve of any example herein, particularly any one of examples 94-99, further comprising a plurality of leaflets secured on an inside of the frame and configured to open and close in order to regulate a flow of blood through the prosthetic heart valve, from the inflow end to an outflow end of the frame, wherein each free apex of the second row of struts is disposed at a level of a portion of the plurality of leaflets that open and close during operation of the prosthetic heart valve

Example 101. The prosthetic heart valve of any example herein, particularly any one of examples 92-100, wherein the frame comprises a plurality of circumferentially extending rows of cells including an outflow row of first cells at least partially defined by the second and third rows of struts and a second row of second cells upstream of the first row of first cells and at least partially defined by the first and second rows of struts, wherein each first cell of the first row of first cells has a first width that is larger than a second width of each second cell of the second row of second cells.

Example 102. The prosthetic heart valve of any example herein, particularly example 101, wherein the first width is twice the second width.

Example 103. The prosthetic heart valve of any example herein, particularly any one of examples 92-102, further comprising an outer skirt disposed around an outer surface of the frame.

Example 104. The prosthetic heart valve of any example herein, particularly any one of examples 92-103, wherein the frame is radially expandable and compressible between a radially expanded configuration and a radially compressed configuration, and wherein the straight edge of the at least one peak is configured to slide upstream on the frame, farther upstream from the respective apex region as the frame is radially compressed and slide downstream on the frame toward but not all the way to the respective apex region.

Example 105. A prosthetic heart valve comprising: a radially expandable and compressible annular frame comprising a plurality of interconnected angled struts defining a plurality of circumferentially extending rows of cells arranged between an outflow end and an inflow end of the frame, wherein the plurality of interconnected angled struts is arranged to form a plurality of circumferentially extending rows of struts, including a first row of struts at the outflow end of the frame, a second row of struts upstream of the first row of struts, and a third row of struts upstream of the second row of struts, wherein the plurality of circumferentially extending rows of cells comprises: a first row of first cells disposed at the outflow end and at least partially defined by the first and second rows of struts and axially extending struts interconnecting struts of the first and second rows of struts; and a second row of second cells disposed upstream of the first row of first cells and at least partially defined by the second and third rows of struts, and wherein the second row of struts comprises a plurality of free apex regions that are not connected to struts of the first row of struts by axially extending struts, and wherein each free apex region connects adjacent ends of a respective pair of angled struts of the second row of struts together and has a first surface facing in a downstream direction, an opposing second surface facing in an upstream direction, and a width measured from the first surface to the second surface, wherein the width is less than width of the struts connected by the free apex region.

Example 106. The prosthetic heart valve of any example herein, particularly example 105, wherein the first surface has a constant convex curvature extending between the adjacent ends of the respective pair of angled struts.

Example 107. The prosthetic heart valve of any of claims 105-106, wherein the first cells are wider than the second cells.

Example 108. The prosthetic heart valve of any of claims 105-107, wherein the second surface forms a depression in the free apex region.

Example 109. The prosthetic heart valve of any example herein, particularly any one of examples 105-108, wherein the second row of second cells has twice the number of cells as the first row of first cells.

Example 110. The prosthetic heart valve of any one of claims 105-109, wherein a portion of the axially extending struts are axially extending window struts that define commissure windows, further comprising a plurality of leaflets secured together at their adjacent sides to form commissures, and wherein the commissures are secured to the commissure windows of the frame.

Example 111. The prosthetic heart valve of any example herein, particularly any one of examples 105-110, wherein the first row of struts form pairs of angled struts, wherein struts of each pair of angled struts of the first row are connected together at their adjacent ends by an outflow apex region, and wherein the outflow apex region curves between the pair of angled struts and has a narrowed width relative to the pair of angled struts.

Example 112. The prosthetic heart valve of any example herein, particularly example 111, wherein the outflow apex region has a third surface and an opposing fourth surface facing in the upstream direction, and wherein the third surface has a constant convex curvature extending between the pair of angled struts.

Example 113. The prosthetic heart valve of any example herein, particularly any one of examples 105-112, wherein each first cell of the first row of first cells has a first axial length that is longer than a second axial length of each second cell of the second row of second cells.

Example 114. The prosthetic heart valve of any example herein, particularly any one of examples 105-113, wherein the frame further comprises a plurality of horizontal struts that extend between adjacent second cells in the second row of second cells, and wherein each horizontal strut of the plurality of horizontal struts connects two adjacent struts of the second row of struts to two adjacent struts of the third row of struts.

Example 115. The prosthetic heart valve of any example herein, particularly example 114, wherein a length, in a circumferential direction, of each horizontal strut is specified to maintain a specified gap between the two adjacent struts of the second row of struts and the two adjacent struts of the third row of struts when the frame is in a radially compressed configuration.

Example 116. The prosthetic heart valve of any example herein, particularly example 115, wherein in the radially compressed configuration, the second row of struts and the third row of struts are oriented axially, in a relatively straight vertical orientation relative to a central longitudinal axis of the frame.

Example 117. The prosthetic heart valve of any example herein, particularly example 116, wherein in the radially compressed configuration, the second row of struts and the third row of struts extend axially, but angle inward toward one another at adjacent horizontal struts.

Example 118. The prosthetic heart valve of any example herein, particularly any one of examples 105-117, wherein each strut in the first row of struts has a first width that is larger than a second width of each strut in the second row of struts.

Example 119. The prosthetic heart valve of any example herein, particularly example 118, wherein the plurality of circumferentially extending rows of struts further includes a fourth row of struts at the inflow end of the frame, and wherein each strut of the fourth row of struts has a third width that is smaller than the first width and larger than the second width.

Example 120. The prosthetic heart valve of any example herein, particularly any one of examples 105-119, further comprising a plurality of leaflets secured on an inside of the frame and configured to open and close in order to regulate a flow of blood through the prosthetic heart valve, from the inflow end to the outflow end of the frame, wherein each free apex region of the second row of struts is disposed at a level of a portion of the plurality of leaflets that open and close during operation of the prosthetic heart valve.

Example 121. The prosthetic heart valve of any example herein, particularly any one of example 105-120, further comprising an inner skirt disposed around an inner surface of the frame, wherein an outflow edge portion of the inner skirt is secured to the second row of struts, and wherein at each free apex region, the outflow edge portion is disposed upstream of the free apex region.

Example 122. The prosthetic heart valve of any example herein, particularly example 121, wherein the outflow edge portion comprises a plurality of peaks spaced apart from each other in a circumferential direction, wherein a portion of the peaks are aligned with respective apex regions and disposed spaced away from the respective apex regions toward an inflow end of the frame.

Example 123. The prosthetic heart valve of any example herein, particularly any one of examples 121 or 122, wherein at each free apex region, the outflow edge portion is folded over against the frame to form a straight edge that is spaced away from and upstream of the free apex region.

Example 124. The prosthetic heart valve of any example herein, particularly any one of examples 121-124, wherein the outflow edge portion of the inner skirt is secured to the second row of struts with a plurality of stitches.

Example 125. A method comprising sterilizing the prosthetic heart valve, apparatus, and/or assembly of any example.

Example 126. A prosthetic heart valve of any one of examples 1-124, wherein the prosthetic heart valve is sterilized.

The features described herein with regard to any example can be combined with other features described in any one or more of the other examples, unless otherwise stated. For example, any one or more of the features of one prosthetic valve frame can be combined with any one or more features of another prosthetic valve frame.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

We claim:

1. A prosthetic heart valve comprising:
a radially expandable and compressible annular frame comprising a plurality of interconnected angled struts defining a plurality of circumferentially extending rows of cells arranged between a first end and a second end of the frame,
wherein the plurality of interconnected angled struts is arranged to form a plurality of circumferentially extending rows of struts, including a first row of struts at the first end of the frame, a second row of struts disposed adjacent to the first row of struts, and a third row of struts disposed adjacent to the second row of struts, the second row of struts disposed between the first and third row of struts,
wherein the plurality of circumferentially extending rows of cells comprises:
a first row of first cells disposed at the first end and at least partially defined by the first and second rows of struts; and
a second row of second cells disposed adjacent to the first row of first cells and at least partially defined by the second and third rows of struts, wherein each first cell of the first row of first cells has a first width that is larger than a second width of each second cell of the second row of second cells, and wherein the second row of struts comprises a plurality of free apex regions, and wherein each free apex region connects adjacent ends of a respective pair of angled struts of the second row of struts together and has a first surface facing the first end of the frame and an opposing second surface facing the second end of the frame, wherein the first surface has a constant convex curvature extending between the adjacent ends of the respective pair of angled struts.

2. The prosthetic heart valve of claim 1, wherein the second surface of each free apex region defines a depressed region between the adjacent ends of the respective pair of angled struts such that a width of the free apex region between the first and second surfaces is less than a width of the respective pair of angled struts.

3. The prosthetic heart valve of claim 1, wherein the first surface of the free apex region forms a single, continuous curve from a surface of a first angled strut of the respective pair of angled struts that faces the first end of the frame and is disposed on a first side of the free apex region to a surface of a second angled strut of the respective pair of angled struts that faces the second end of the frame and is disposed on an opposite, second side of the free apex region.

4. The prosthetic heart valve of claim 1, wherein the first width is two times wider than the second width.

5. The prosthetic heart valve of claim 1, wherein the frame further comprises a plurality of axially extending struts that extend between the first row of struts and the second row of struts and define axial sides of the first row of first cells.

6. The prosthetic heart valve of claim 5, wherein a first portion of struts of the second row of struts form pairs of angled struts that are each connected to a respective axially extending strut of the plurality of axially extending struts, wherein a second portion of struts of the second row of struts form pairs of angled struts that are connected by a respective free apex region of the plurality of apex regions, and wherein the plurality of free apex regions are unattached to the plurality of axially extending struts.

7. The prosthetic heart valve of claim 5, wherein a portion of the plurality of axially extending struts are axially extending window struts that define commissure windows, further comprising a plurality of leaflets secured together at their adjacent sides to form commissures, and wherein the commissures are secured to the commissure windows of the frame.

8. The prosthetic heart valve of claim 1, wherein the first row of struts form pairs of angled struts, wherein the struts of each pair of angled struts are connected together at their adjacent ends by an outflow apex region, and wherein the outflow apex region curves between the pair of angled struts and has a narrowed width relative to the pair of angled struts.

9. The prosthetic heart valve of claim 1, wherein each first cell of the first row of first cells has a first axial length that is longer than a second axial length of each second cell of the second row of second cells.

10. The prosthetic heart valve of claim 1, wherein the frame further comprises a plurality of horizontal struts that extend between adjacent second cells in the second row of second cells, and wherein each horizontal strut of the plurality of horizontal struts connects two adjacent struts of the second row of struts to two adjacent struts of the third row of struts.

11. The prosthetic heart valve of claim 1, further comprising a plurality of leaflets secured on an inside of the frame and configured to open and close in order to regulate a flow of blood through the prosthetic heart valve, from the second end to the first end of the frame, wherein each free apex region of the second row of struts is disposed at a level of a portion of the plurality of leaflets that open and close during operation of the prosthetic heart valve.

12. The prosthetic heart valve of claim 1, wherein the first end is an outflow end of the frame, and the second end is an inflow end of the frame.

13. The prosthetic heart valve of claim 1, further comprising an inner skirt disposed around an inner surface of the frame, wherein an outflow edge portion of the inner skirt is secured to the second row of struts, and wherein at each free apex region, the outflow edge portion is disposed upstream of the free apex region.

14. A prosthetic heart valve comprising:
a radially expandable and collapsible annular frame comprising a plurality of interconnected struts defining a plurality of circumferentially extending rows of cells arranged between an outflow end and an inflow end of the frame, wherein the plurality of interconnected struts comprises:
a circumferentially extending row of first struts defining the outflow end, each first strut comprising two angled strut portions interconnected by an outflow apex region, wherein the outflow apex region curves between the two angled strut portions and has a narrowed width relative to a width of the two angled struts portions;
a plurality of axially extending struts spaced circumferentially apart around the frame and connected to the row of first struts;
a circumferentially extending row of angled second struts disposed upstream of the row of first struts, wherein a first portion of second struts of the row of angled second struts are each directly connected to a respective axially extending strut of the plurality of axially extending struts, and wherein a second portion of second struts of the row of angled second struts form pairs of second struts which are connected together by a free apex region that is unattached to the plurality of axially extending struts, wherein the free apex region of each respective pair of second struts has a first surface facing in a downstream direction and an opposing second surface facing in an upstream direction, wherein the first surface curves between the respective pair of second struts, and wherein the second surface depresses inward toward the first surface such that a width of the free apex region between the first and second surfaces is less than a width of the respective pair of second struts; and
a circumferentially extending row of angled third struts, wherein the row of first struts, the axially extending struts, and the row of angled second struts form a first row of cells of the plurality of rows of cells disposed at the outflow end, wherein the row of angled second struts and the row of angled third struts form a second row of cells of the plurality of rows of cells that are disposed adjacent to the first row of cells, and wherein a first width of each cell of the first row of cells is wider than a second width of each cell of the second row of cells; and
a plurality of leaflets secured on an inside of the frame and configured to open and close in order to regulate a flow of blood through the prosthetic heart valve, from the inflow end to the outflow end of the frame, wherein each free apex region of the row of angled second struts is disposed at a level of a portion of the plurality of leaflets that open and close during operation of the prosthetic heart valve.

15. The prosthetic heart valve of claim 14, wherein the first surface of each free apex region has a constant convex curvature between downstream facing surfaces of the respective pair of second struts.

16. The prosthetic heart valve of claim 14, wherein a first surface of the outflow apex region of each first strut that faces away from the inflow end of the frame forms a single, continuous curve with a convex curvature from one angled strut portion of the two angled strut portions on a first side of the outflow apex region to another angled strut portion of the two angled strut portions on a second side of the outflow apex region.

17. The prosthetic heart valve of claim 14, wherein the first width is two times wider than the second width.

18. A prosthetic heart valve comprising:
a radially expandable and compressible annular frame comprising a plurality of interconnected struts defining a plurality of circumferentially extending rows of cells arranged between an inflow end and an outflow end of the frame, the plurality of interconnected struts comprising:
a circumferentially extending row of outflow struts defining the outflow end, wherein each outflow strut comprises two angled strut portions interconnected by an apex region, wherein each apex region curves between a corresponding pair of two angled strut portions and has a narrowed width and a length that extends along at least 25% of a total length of the outflow strut, wherein the narrowed width is smaller than a width of the two angled strut portions; and
a circumferentially extending row of angled first struts disposed upstream of the row of outflow struts, wherein the row of outflow struts and the row of angled first struts at least partially form a first row of cells of the plurality of circumferentially extending rows of cells disposed at the outflow end, and wherein each cell of the first row of cells has a first width that is larger than a second width of cells of remaining rows of cells of the plurality of circumferentially extending rows of cells.

19. The prosthetic heart valve of claim 18, wherein the plurality of interconnected struts further comprises a circumferentially extending row of angled second struts disposed upstream of the row of angled first struts, and wherein the row of angled first struts and the row of angled second struts form a second row of cells of the plurality of rows of cells that is disposed adjacent to and upstream of the first row of cells, and wherein each cell of the second row of cells has the second width which is half the first width.

20. The prosthetic heart valve of claim 19, wherein a portion of first struts of the row of angled first struts form pairs of first struts which are connected together by a free apex region that is unattached to additional struts forming the first row of cells, wherein the free apex region of each respective pair of second struts has a first surface facing in a downstream direction and an opposing second surface facing in an upstream direction, wherein the first surface curves between the respective pair of second struts, and wherein the second surface depresses inward toward the first surface such that a width of the free apex region between the first and second surfaces is less than a width of the respective pair of second struts.
